(19) United States
(12) Patent Application Publication
(10) Pub. No.: US 2025/0130244 A1
(43) Pub. Date: Apr. 24, 2025

(54) METHOD FOR DIAGNOSING ENDOMETRIOSIS AND FOR CLASSIFYING THE STAGE OF ENDOMETRIOSIS

(71) Applicant: Roche Diagnostics Operations, Inc., Indianapolis, IN (US)

(72) Inventors: Aljoscha Michael Flohr, Bremen (DE); Aikaterini Georgopoulou, Luzern (CH); Rym Guennoun, Luzern (CH); Martin Hund, Horw (CH); Martin Klammer, Munich (DE)

(21) Appl. No.: 18/999,357

(22) Filed: Dec. 23, 2024

Related U.S. Application Data
(63) Continuation of application No. PCT/EP2023/067108, filed on Jun. 23, 2023.

(30) Foreign Application Priority Data
Jun. 23, 2022 (EP) 22180800.9

(51) Int. Cl. G01N 33/68

(52) U.S. Cl. CPC G01N 33/6893; G01N 2333/70596; G01N 2800/364; G01N 2800/52; G01N 2800/56

(57) ABSTRACT

The present invention relates to methods of diagnosing whether a subject has endometriosis, to methods of classifying the stage of endometriosis, to methods of determining the therapeutic effect of a treatment regimen for endometriosis, and methods of monitoring endometriosis progression in a subject, by determining the amount or concentration of c-Kit in a sample of the subject, and comparing the determined level to a reference value.

The present invention relates to methods of diagnosing whether a subject has endometriosis, methods of assessing the risk of a subject having endometriosis, to methods of classifying the stage of endometriosis, to methods of determining the therapeutic effect of a treatment regimen for endometriosis, and methods of monitoring endometriosis progression in a subject, by determining the amount or concentration of c-Kit in a sample of the subject, and comparing the determined level to a reference value.

### BACKGROUND OF THE INVENTION

Endometriosis is a chronic disorder defined by the growth of endometrial glands and stroma-like lesions outside the uterus (Liu et al., 2011). The lesions can be peritoneal lesions, superficial implants or cysts on the ovary, or deep infiltrating disease. It arises from eutopic endometrial cells characterized by increased proliferation and adhesion properties (Liu et al., 2011). The increased cell viability in eutopic endometrium is a consequence of reducing apoptosis and an increase in cell proliferation (Johnson et al., 2005). Endometriosis affects 5-8% of all women of reproductive age and 70% of women with chronic pelvic pain.

The prevalence of endometriosis has been estimated at 176 million women worldwide (Adamson et al. J Endometr. 2010; 2:3-6). For many of these women there is often a delay in diagnosis of endometriosis resulting in unnecessary suffering and reduced quality of life. In patients aged 18-45 years, there is a delay of 7-10 years. As most women with endometriosis report the onset of symptoms during adolescence, early referral, diagnosis, identification of disease and treatment may mitigate pain preventing disease progression. Barriers to early diagnosis include the high cost of diagnosis and treatment in adolescent patients and presentation of confounding symptoms such as cyclic and non-cyclic pain (Parasar et al. Curr Obstet Gynecol Rep. 2017; 6:34-41).

Gold standard for the diagnosis of endometriosis is laparoscopic visualization and subsequent histological confirmation. Until now, there are no non-invasive methods for the diagnosis of endometriosis (Hsu et al. Clin Obstet Gynecol 2010:53: 413-419). During a diagnostic laparoscopy, a gynaecologist with training and skills in laparoscopic surgery for endometriosis should perform a systematic inspection of the pelvis (NICE guideline NG73, 2017). Surgical visualization requires good expertise, training and skills for reliable diagnosis. The fact that laparoscopic surgery is needed for diagnosis, which is avoided by doctors as long as possible, leads to a 7-10 year delay in diagnosis. The lack of a non-invasive diagnostic test significantly contributes to the long delay between onset of the symptoms and definitive diagnosis of endometriosis (Signorile and Baldi. J Cell Physiol 2014; 229:1731-1735). Thus, there is an unmet medical need for a non-invasive test for the diagnosis of endometriosis, in particular for the diagnosis of early, minimal and mild endometriosis (revised American Society for Reproductive Medicine rASRM stages I-II).

Non-invasive diagnosis of endometriosis would allow earlier diagnosis and treatment, with the potential to improve quality of life and reduce the societal costs related to endometriosis, and has therefore been selected as a research priority by the World Endometriosis Society (WES) and the World Endometriosis Research Foundation (WERF) (Fassbender et al., Springer, Peripheral Blood Biomarkers for Endometriosis. 2017). Thus, a non-invasive tool to diagnose endometriosis could facilitate earlier diagnosis and intervention that could ultimately improve quality of life and preserve fertility (Parasar et al. Curr Obstet Gynecol Rep. 2017; 6:34-41).

Blood biomarkers are essential to reduce the time delay of diagnosing endometriosis that require laparoscopy. CA-125 is one of the most commonly used blood biomarkers, however, its diagnostic utility is limited to endometriosis rASRM stages III and IV (Nisenblat et al., Cochrane Database of Systematic Reviews. 2016; 5: CD012179).

c-Kit is a proto-oncogene encoding a 145 kd transmembrane tyrosine kinase receptor (CD117) (Roskoski et al., 2005). Stem cell factor (SCF) is a cognate ligand of the c-Kit receptor. SCF-induced c-Kit signaling has been shown to play crucial roles in a diverse range of biological functions (Sharkey et al., 1994). Serum levels of c-Kit increase in proliferative mast cell disorders, suggesting the existence of c-Kit shedding pathways in mast cells (Cruz et al. J Biol Chem. 2004). It is essential for the survival, differentiation, and mobilization of multiple cell types, including myeloid, erythroid, megakaryocytic, lymphoid, germ cell and melanocyte progenitors (Besmer et al., 1991, Lyam et al., 1998, Papayannopoulou et al., 1991). Several studies have highlighted that the dysregulation of c-Kit function is associated with the development of various diseases, including cancer (Vliagoftis et al., 1997, Miettinen et al., 2005). For example, in mast cells, where c-Kit regulates differentiation, activation and homeostasis (Gilfillan et al., 2011), mutations within the c-Kit receptor can interfere with the signalling cascade and induce constitutive receptor activation independent of SCF, leading to mastocytosis (Cardet et al., 2013). Furthermore, SCF binding to c-Kit in endothelial cells disrupts the endothelial adherens junctions enhancing vascular leaking (Kim et al., 2014). Alteration of c-Kit signalling has also been linked with oncogenesis and the development of a wide variety of benign and malignant neoplastic disorders, including gastro-intestinal-stromal tumours (GISTs), acute myeloid leukaemia (AML), mast cell leukaemia (MCL), and melanomas (Ha et al., 2010, Matsuda et al., 1993, Sakurai et al., 1999, Ayatollahi et al., 2017, Boissan et al., 2000, Montone et al., 1997).

The levels/presence of a biomarker can differ when measured in the tissue or in blood serum. For example, the complement component C7 and complement component C4showed overexpression in ectopic endometrium of women with endometriosis compared to eutopic endometrium of control women without endometriosis (Ahn et al. Fertil Steril 2016; Eyster et al. Fertil Steril 2007). However, no increase of serum complement component C7 protein nor complement component C4 protein was found in circulating blood (Hever et al. PNAS 2007).Brain-derived neurotrophic factor (BDNF) mRNA expression levels were higher in ovarian endometriotic lesions than in eutopic endometrium (Wang et al. Journal of Ovarian Research 2022). However, serum BDNF was not significantly different in women with endometriosis compared to control women without endometriosis (Perricos et al. Exp Biol Med (Maywood) 2018). Furthermore, in breast cancer, while it has observed that molecular markers such as CEA (O), ERβ, CK19 and, c-Myc were significantly different between blood of normal and patients while there were no significant differences of these markers in tissue samples.

Therefore, the locally altered expression of biomarkers in tissue does not translate 1:1 to significantly different levels of these biomarkers in circulating blood.

There is a high need for a non-invasive diagnosis of endometriosis by using a biomarker, which allow a reliable and early risk assessment and/or identification of patients exhibiting signs and symptoms of endometriosis.

The present invention, therefore, provides means and methods complying with these needs.

### SUMMARY OF THE INVENTION

In a first aspect, the present invention relates to an in vitro method for diagnosing endometriosis in a subject, the method comprising the steps of:
a) determining the level of c-Kit in a biological fluid sample from the subject,
b) comparing the level of c-Kit to at least one appropriate reference value of a c-Kit level,
c) identifying a subject as having endometriosis if the comparison in step b) indicates that the subject has an increased level of c-Kit compared to the appropriate reference value.

In a second aspect the present invention relates to an in vitro method for classifying the stage of endometriosis in a subject, the method comprising the steps of:
a) determining the level of c-Kit in a biological fluid sample from the subject,
b) comparing the level of c-Kit to at least one appropriate reference value of a c-Kit level,
c) classifying the stage of endometriosis in the subject if the comparison in step b) indicates that the subject has an increased or decreased level of c-Kit compared to the at least one appropriate reference value of a c-Kit level.

In a third aspect, the present invention relates to in vitro method for monitoring endometriosis progression in a subject, the method comprising the steps of:
i. determining the level of c-Kit in a biological fluid sample from the subject in accordance with method steps a) to b) described above for diagnosing and classifying endometriosis,
ii. repeating step i. using a biological fluid sample obtained from the subject during or after treatment for a time interval; and
iii. comparing the levels of c-Kit identified in i. with the c-Kit levels identified in ii., wherein a change in the c-Kit levels from i. to ii. is indicative of a change in endometriosis progression in the subject.

In a fourth aspect the present invention relates to an in vitro method for determining the therapeutic effect of a treatment regimen for endometriosis in a subject, the method comprising the steps of:
i. determining the level of c-Kit in a biological fluid sample from the subject in accordance with method steps a) to b) described above for diagnosing and classifying endometriosis,
ii. repeating step i. using a biological fluid sample obtained from the subject during or after treatment for a time interval; and
iii. comparing the level of c-Kit determined in step i. to that determined in step ii., and identifying that the treatment regimen has a therapeutic effect if the level of c-Kit decreased after treatment.

### BRIEF DESCRIPTION OF THE FIGURES

Embodiments of the invention are further described hereinafter with reference to the accompanying drawings.

FIG. 1: Box plot analysis: c-Kit levels [ng/mL] in serum samples of control groups (non-pathological controls, symptomatic controls (no benign findings) versus endometriosis case groups (stage I, endometriosis stage II, endometriosis stage III and endometriosis stage IV) and controls with benign findings. Serum c-Kit levels were measured using human CD117/c-Kit Quantikine ELISA Kit (R&D Systems, USA).

FIG. 2 (A and B): Boxplot analysis: c-Kit levels [ng/mL] in serum samples of women with endometriosis (stages I-II-III-IV) compared to controls without endometriosis. c-Kit (A) and CA-125 (MUC16) (B) levels were measured using Olink proteomics technology. A: N negative (Ctrl): 16; N positive (Endo): 24; AUC 0.77 (0.62-0.92); B: N negative (Ctrl): 16; N positive (Endo): 24; AUC 0.76 (0.61-0.92)

### DETAILED DESCRIPTION OF THE INVENTION

The invention is based on the surprising finding that elevated c-Kit levels in serum is associated with endometriosis. C-Kit levels are increased at all stages I, II, III and IV whereby stage II shows the highest level of c-Kit and stages I, III and IV show a comparable increased level, which confers serum c-Kit a diagnostic potential for early detection of endometriosis.

The inventors show for the first time that c-Kit measured in serum is increased in women with endometriosis compared to controls. C-Kit levels are specifically increased in endometriosis stages I and in particular, stage II (minimal/mild endometriosis).

There is an unmet medical need for a non-invasive test for the reliable diagnosis and/or classification of endometriosis, and in particular of early endometriosis. C-Kit has the advantage of a non-invasive blood-based test that identifies women with early endometriosis.

The inventors have investigated the levels of c-Kit in serum obtained from women with endometriosis. Surprisingly, they found that an increased level of c-Kit can be detected in serum samples from women with endometriosis. In particular, the fact that already in women with early stages of endometriosis increased c-Kit levels can be detected make this marker to a helpful tool to diagnose endometriosis at early stages. Assays that enable the determination of the level of c-Kit in such biological fluids may therefore be useful for endometriosis diagnosis and/or classification, prognosis and patient stratification for treatment.

The data presented herein show that determination of c-Kit levels in serum provides a means for diagnosing endometriosis, risk stratification of having endometriosis, and classifying the stage of endometriosis in a subject (for example, when determining the c-Kit levels at regular intervals in the subject or by comparing the determined value to a value of a known stage). This also allows the monitoring of endometriosis progression and/or the evaluation of treatment regimen.

The data presented herein also show that determination of c-Kit levels in serum provides a means for detecting early stages of endometriosis and control samples more accurately than CA-125.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, levels, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In general, the methods described are in vitro methods that are performed using a sample that has already been obtained from the subject (i.e., the sample is provided for the method, and the steps taken to obtain the sample from the subject are not included as part of the method). The methods may therefore include the step of providing a biological fluid sample from a subject.

As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample).

The methods provided herein comprise providing a biological fluid sample (for example a blood sample) from a subject. The samples being tested in the methods described herein are also referred to as “test samples”.

As used herein, the terms “biological (fluid) sample”, “test sample”, “sample” are used interchangeably, and variations thereof refer to a sample obtained or derived from a subject. For the purposes described herein, the sample is, or comprises, a biological fluid (also referred to herein as a bodily fluid) sample.

Examples of samples include but are not limited to fluid samples such as blood, serum, plasma, synovial fluid, interstitial fluid, capillary blood, peritoneal fluid, menstrual fluid, urine, saliva, and lymphatic fluid. Analysis of a sample may be accomplished on chemical basis. Chemical analysis includes but is not limited to the detection of the presence or absence of specific indicators or alterations in their amount, concentration or level.

The sample is an in vitro sample, it will be analyzed in vitro and not transferred back into the body.

A blood sample may be a whole blood sample, or a processed blood sample e.g., serum, plasma etc. Methods for obtaining biological fluid samples (e.g., whole blood, serum, plasma, etc) from a subject are well known in the art. For example, methods for obtaining blood samples from a subject are well known and include established techniques used in phlebotomy. The obtained blood samples may be further processed using standard techniques to obtain e.g., a serum sample, or a plasma sample. Advantageously, methods for obtaining biological fluid samples from a subject are typically low-invasive or non-invasive.

A whole blood sample is defined as a blood sample drawn from the body and from which (substantially) no constituents (such as platelets or plasma) have been removed. In other words, the relative ratio of constituents in a whole blood sample is substantially the same as a blood in the body. In this context, “substantially the same” allows for a very small change in the relative ratio of the constituents of whole blood e.g., a change of up to 5%, up to 4%, up to 3%, up to 2%, up to 1% etc. Whole blood contains both the cell and fluid portions of blood. A whole blood sample may therefore also be defined as a blood sample with (substantially) all of its cellular components in plasma, wherein the cellular components (i.e., at least comprising the requisite white blood cells, red blood cells, platelets of blood) are intact.

In a preferred example, the biological fluid sample is serum.

Methods for analysing (and optionally isolating, enriching for or extracting) protein biomarkers from blood, plasma, serum, saliva, and urine samples have been described previously, see for example, Heitzer, E., Haque, I. S., Roberts, C. E. S. et al. Current and future perspectives of liquid biopsies in genomics-driven oncology. Nat Rev Genet 20, 71-88 (2019).

In the context of present invention, the term “biomarker” refers to a substance within a biological system that is used as an indicator of a biological state of said system. In the art, the term “biomarker” is sometimes also applied to means for the detection of said endogenous substances (e.g. antibodies, nucleic acid probes etc, imaging systems). In the context of present invention, the term “biomarker” shall be only applied for the substance, not for the detection means. Thus, biomarkers can be any kind of molecule present in a living organism, such as a nucleic acid (DNA, mRNA, miRNA, rRNA etc.), a protein (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA) or a substance that has been internalized by the organism or a metabolite of such a substance. A biomarker is an organic biomolecule (e.g., a protein, polypeptide, peptide, isomeric form thereof, immunologically detectable fragment thereof, corresponding nucleic acid molecule (e.g., mRNA, cDNA etc)) which is differentially present in a sample taken from a subject having a disease as compared with a subject not having the disease. A biomarker is differentially present if the mean or median level of the biomarker in the different groups is calculated to be statistically relevant. Common tests for statistical significance include, among others, t-test (e.g., student t-test), ANOVA, Kruskal-Wallis, Wilcoxon, Mann-Whitney, Receiver Operating Characteristic (ROC curve), accuracy and odds ratio. Biomarkers, alone or in combination, provide measures of relative risk that a subject belongs to one phenotypic status or another.

Therefore, they are useful as markers for disease (diagnostics), therapeutic effectiveness of a drug and drug toxicity.

Typically, the biomarker referred to herein is measured at the protein level.

The methods provided herein refer to “determining” the level of one or more proteins. As would be clear to a person of skill in the art, the level of one or more proteins is typically “determined” by measuring the level of the protein in the sample. The term “determining” can therefore be replaced with the term “measuring” or “determining by measuring” herein.

The terms “determining” or “assessing” as used herein also refer to assessing/determining whether a patient suffers from endometriosis. Accordingly, assessing/determining as used herein includes diagnosing endometriosis, assessing the risk that a subject suffers from endometriosis, selecting for therapy of endometriosis, monitoring a patient suffering from endometriosis or being treated for endometriosis, by determining the amount or concentration of c-Kit in a sample of the patient, and comparing the determined amount or concentration to a reference. Typically, the assessment referred to in accordance with the present invention is the assessment of the presence of endometriosis

The term “measurement”, “measuring” or “determining” preferably comprises a qualitative, a semi-quantitative or a quantitative measurement.

Conventional “determining” methods may include sending a clinical sample(s) to a commercial laboratory for measurement of the biomarker levels in the biological fluid sample, or the use of commercially available assay kits for measuring the biomarker levels in the biological fluid sample. Exemplary kits and suppliers will be apparent to a person of skill in the art. In various examples, biomarkers may be determined, detected and/or quantified using ELISA assays or lateral flow devices, such as for point-of-care use, as well as spot check colorimetric tests.

The terms “level” or “amount” as used herein encompass the absolute amount of a biomarker as referred to herein, the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts measured from biological read out systems in response to the peptides or intensity signals obtained from specifically bound ligands. It is to be understood that values correlating to the aforementioned amounts or parameters can also be obtained by all standard mathematical operations.

The level of biomarker present in the biological fluid sample may be determined by e.g. assaying the amount of protein biomarker present in the sample. Assays for measuring the amount of a specified protein are well known in the art and include direct or indirect measures. The level of protein biomarker in a sample may also be determined by determining the level of protein biomarker activity in a sample. Accordingly, protein “level” encompasses both the amount of protein per se, or its level of activity.

By way of example, the level of a protein biomarker in a biological fluid sample can be determined (e.g., measured) by any suitable methods and materials known in the art, including, for example, a process selected from the group consisting of mass spectrometry, immunoassays, enzymatic assays, spectrophotometry, colorimetry, fluorometry, bacterial assays, protein microarrays, compound separation techniques, or other known techniques for determining the presence and/or quantity of an analyte. Examples of relevant techniques include enzyme linked immunosorbent assays (ELISAs), immunoprecipitation, immunofluorescence, enzyme immunoassay (EIA), radioimmunoassay (RIA), Western blot analysis, and Lateral Flow (using e.g. Lateral Flow Devices (LFDs) utilizing a membrane bound antibody specific to the protein biomarker).

Preferably, the level of a protein biomarker in a biological fluid sample is measured by ELISA or lateral flow.

The term “at regular intervals” as used herein refers to the periodical determination of the c-Kit levels in the same subject after a predetermined time. Since every subject is different the speed of development of the disease may vary from subject to subject. Whereas in one subject disease progression can progress through from stage I to stage IV within a few years it might stagnate at one stage for several years in another subject. Therefore, to obtain a continuous picture of the development of the c-Kit levels in a subject and to be able to allocate the obtained c-Kit levels to the corresponding disease stages I, II, III and IV the intervals should be chosen in a way that no stages of disease progression are skipped. Preferably the c-Kit levels of a subject are determined every 6, 7, 8, 9, 10, 11, or 12 months.

The term “c-Kit” is the gene encoding the receptor tyrosine kinase protein CD117, also known as c-kit and SCF R. It is the cellular receptor for Stem Cell Factor (SCF), also named c-kit ligand, mast cell growth factor, and steel factor. This receptor-ligand system plays essential roles in germ cell development, melanogenesis, hematopoiesis, and oncogenesis. CD117 is expressed on lymphoid, myeloid, erythroid, and megakaryocyte progenitors, NK cells, germ cells, melanocytes, glial cells, vascular smooth muscle cells, placenta, and epithelial cells. Altered levels and mutations in CD117 are associated with several types of cancer including lung, breast, gastrointestinal stromal, and germ cell tumors. Multiple transcript variants encoding different isoforms have been found for this gene. SCF exerts its biologic effect through binding to the receptor c-Kit, which in turn is expressed in mast cells, hematopoietic stem cells and cells of the germ line [Cho N H. et al. (2004) Fertil Steril 81:403-7]. The amino acid sequence of human c-Kit can be accessed via UniProt (see UniProtKB —P10721 (KIT_HUMAN)_HUMAN). There are three isoforms described for c-Kit with the identifiers P10721-1, P10721-2, and P10721-4.

“CA-125”, the Carbohydrate antigen 125, sometimes named as Cancer Antigen 125 or Tumor Antigen 125, is a mucin-type glycoprotein, produced by the MUC16 gene, and associated with the cellular membrane. CA-125 is a biomarker for epithelial cell ovarian cancer being derived from coelomic epithelia including the endometrium, fallopian tube, ovary, and peritoneum. Diagnostic use of CA-125 is limited to endometriosis stages III and IV (moderate and severe endometriosis) with moderate sensitivity.

“Symptoms” of a disease are implications of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include but are not limited to an unpleasant sensation that may be felt as a persistent or varying burning, throbbing, itching or stinging ache.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to inflammatory diseases, infectious diseases, cutaneous conditions, endocrine diseases, intestinal diseases, neurological disorders, joint diseases, genetic disorders, autoimmune diseases, traumatic diseases, and various types of cancer.

“Endometriosis” is a chronic, hormone-dependent, inflammatory disease that is characterized by lesions of endometrial-like tissue outside of the uterus. Clinical presentation of endometriosis varies significantly from patient to patient. Endometriosis patients often present with symptoms such as intermenstrual bleeding, painful periods (dysmenorrhea), painful intercourse (dyspareunia), painful defecation (dyschezia) and painful urination (dysuria). Pelvic pain due to endometriosis is usually chronic (lasting ≥6 months) and is associated with dysmenorrhea (in 50 to 90% of cases), dyspareunia, deep pelvic pain, and lower abdominal pain with or without back and loin pain. The pain can occur unpredictably and intermittently throughout the menstrual cycle or it can be continuous, and it can be dull, throbbing, or sharp, and exacerbated by physical activity. Bladder- and bowel-associated symptoms (nausea, distention, and early satiety) are typically cyclic. Pain often worsens over time and may change in character; infrequently, women report burning or hypersensitivity, symptoms that are suggestive of a neuropathic component. Often, endometriosis can be asymptomatic, only coming to a clinician's attention during evaluation for infertility (Sinaii et al. Fertil Steril. 2008; 89 (3): 538-545). In women with endometriosis, there is a reduced monthly fecundity rate (2-10%) compared with fertile couples (15-20%). Although endometriosis impairs fertility, it does not usually completely prevent conception (Fadhlaoui et al. Front Surg. 2014; 1:24).

The most commonly affected sites of endometriosis are the pelvic organs and peritoneum, although other parts of the body such as the lungs are occasionally affected. The extent of the disease varies from a few, small lesions on otherwise normal pelvic organs to large, ovarian endometriotic cysts (endometriomas) and/or extensive fibrosis and adhesion formation causing marked distortion of pelvic anatomy. Based on the location, endometriotic lesions can be classified into peritoneal endometriosis, ovarian endometriotic cysts (endometrioma), deep nodules (deep infiltrating endometriosis), and adenomyosis (Kennedy et al. Hum Reprod. 2005; 20 (10): 2698-2704). Deep infiltrating endometriosis is considered to be any manifestation of endometriosis that is located other than in the superficial tissues of the rectovaginal septum and vaginal fornix, the pelvic wall, parametrium, bowel, uterus, or urinary bladder (Halis. et al. (2010). Deutsches Ärzteblatt International, 107 (25), 446). Endometriosis can also involve the diaphragm (diaphragmatic endometriosis) or involve the thorax (thoracic endometriosis) (Nezhat et al. JSLS 2019).

The term “rASRM stage” or “rASRM staging” refers to the revised classification system established by the American Society for Reproductive Medicine (ASRM) describing the severity of endometriosis based on the findings at surgery (laparoscopy). The classification is based on the morphology of peritoneal and pelvic implants such as red, white and black lesions, percentage of involvement of each lesion should be included. Number, size, and location endometrial implants, plaques, endometriomas and adhesions should be noted. Endometriosis in bowel, urinary tract, fallopian tube, vagina, cervix, skin, or other locations should be documented per ASRM guidelines. Stages of endometriosis according to ASRM guidelines are stage I, II, III, and IV determined based on the point scores and correspond to minimal, mild, moderate and severe endometriosis. The rASRM stages I & II endometriosis (minimal to mild endometriosis) are defined by superficial peritoneal endometriosis, possible presence of small deep lesions, absence of endometrioma and/or mild filmy adhesion. The rASRM stages III and IV endometriosis (moderate to severe endometriosis) are defined by the presence of superficial peritoneal endometriosis, deep infiltrating endometriosis with moderate to extensive adhesions between the uterus and bowels and/or endometrioma cysts with moderate to extensive adhesions involving the ovaries and tubes.

The term “VAS”, the Visual Analog Scale, is an instruments to assess the intensity of pain. The VAS consists of a 10-cm long horizontal line with its extremes marked as ‘no pain’ and ‘worst pain imaginable’. Each patient ticks her pain level on the line and the distance from ‘no pain’ on the extreme left to the tick mark is measured in centimeters, yielding a pain score from 0 to 10. ‘No pain’ corresponds to a pain score of 0, ‘worst pain imaginable’ corresponds to a pain score of 10. In women with endometriosis dysmenorrhea is associated with the highest perception of pain with a mean VAS score of about 6 (Cozzolino et al. Rev Bras Ginecol Obstet 2019; 41 (3): 170-175).

The subject may be referred to herein as a patient. The terms “subject”, “individual”, and “patient” are used herein interchangeably and refer to an animal, preferably a mammal and, more typically to a human. The patient is preferably a human female. There is a need for diagnosis of endometriosis at a young age, as it starts with the initiation of the menstruation. Therefore, the patient is preferably a young or adolescent human female aged between 12-24 years. In an embodiment of the present invention, the patient is a young or adolescent human female. The subject can be symptomatic (e.g., the subject presents symptoms associated with endometriosis), or the subject can be asymptomatic (e.g., the subject does not present symptoms associated with endometriosis). The subject may be diagnosed with, be at risk of developing or present with symptoms of endometriosis. The subject may have, or be suspected of having (e.g., present with symptoms or a history indicative or suggestive of), endometriosis.

Accordingly, in some examples, the subject has endometriosis (and the method diagnoses, identifies, (or detects) that the subject has endometriosis). In this context, the terms “diagnose” “identify”, and “detect” can be used interchangeably.

In particular examples, the subject has early stage (stage I or stage II) endometriosis.

The patient to be investigated by the method of the present invention shall be a patient having suspected endometriosis. The term “suspected endometriosis” as used herein means that the patient shall exhibit clinical parameters, signs and/or symptoms of endometriosis. Thus, the patient according to the invention is, typically, a patient that suffers from an endometriosis or is suspected to suffer from an endometriosis. A patient having a suspicion of endometriosis with signs and symptoms: dysmenorrhea (painful menstrual periods), dysuria (painful urination), dyschezia (difficult or painful defecation), dyspareunia (pain during or after sexual intercourse) and chronic abdominal/pelvic pain independent of menstrual cycle, heavy menstrual bleeding, long menstrual periods, infertility, fatigue, cyclical lung problems (pneumothorax), cyclical cough, chest pain, or coughing of blood (haemoptysis), shoulder tip pain, painful rectal bleeding or the presence of blood in the urine (haematuria), and cyclical scar swelling and pain (EHRE Information on Endometriosis, 2022 www.eshre.eu/guidelines).

Alternatively, the c-Kit levels are determined routinely as part of screening tests without any suspicion on endometriosis but to be able to detect symptom-free endometriosis at early stages.

By detecting increased levels of c-Kit in a subject the suspicion that the subject suffers from endometriosis would be confirmed and there is a high risk that that the subject suffers from endometriosis. In particular, in cases where the subject already exhibits clinical parameters, signs and/or symptoms of endometriosis the determination of increased c-Kit levels would confirm the presence of endometriosis.

The term “comparing” as used herein refers to comparing the amount/level of the biomarker in the sample from the subject with the reference amount or reference value of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer assisted. Thus, the comparison may be carried out by a computing device. The value of the measured or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provides the desired assessment in a suitable output format.

The terms “(appropriate) reference value”, “reference sample” or “control (sample)” as used herein, refers to a sample which is analysed in a substantially identical manner as the sample of interest and whose information is compared to that of the sample of interest. A reference sample thereby provides a standard allowing for the evaluation of the information obtained from the sample of interest. A control sample may be derived from a body fluid of a healthy individual, in particular serum or plasma for a non-invasive test, thereby providing a standard of a healthy status of a tissue, organ or individual. Differences between the status of the normal reference sample and the status of the sample of interest may be indicative of the presence or further progression of such disease or disorder. A control sample may be derived from an abnormal or diseased tissue, organ or individual thereby providing a standard of a diseased status of a tissue, organ or individual. Differences between the status of the normal or abnormal reference sample and the status of the sample of interest may be indicative of the absence or bettering of such disease or disorder.

A reference sample may also be derived from the same tissue, organ, or individual as the sample of interest but has been taken at an earlier time point. Differences between the status of the earlier taken reference sample and the status of the sample of interest may be indicative of the progression of the disease, i.e. a bettering or worsening of the disease over time and by this may allow to classify the stage of endometriosis of being stage I, II, III or IV.

The determined value can be compared to more than one (appropriate) reference values, which can be of different kind. For example the determined value can be compared to one or more values obtained from the same subject at earlier time points and in parallel it can be compared to one or more values obtained from other subjects (with a known stage of endometriosis).

The control sample may be an internal or an external control sample. An internal control sample is used, i.e. the marker level(s) is (are) assessed in the test sample as well as in one or more other sample(s) taken from the same subject to determine if there are any changes in the level(s) of said marker(s). For an external control sample the presence or amount of a marker in a sample derived from the individual is compared to its presence or amount in an individual known to suffer from, or known to be at risk of, a given condition; or an individual known to be free of a given condition, i.e., “normal individual”.

It will be appreciated by the skilled artisan that such external control sample may be obtained from a single individual or may be obtained from a reference population that is age-matched and free of confounding diseases. Typically, samples from 100 well-characterized individuals from the appropriate reference population are used to establish a “reference value”. However, reference population may also be chosen to consist of 20, 30, 50, 200, 500 or 1000 individuals. Healthy individuals represent a preferred reference population for establishing a control value.

For example, a marker concentration in a patient sample can be compared to a concentration known to be associated with a specific course of a certain disease. For example, it can be compared to a concentration known to be associated with a certain stage of endometriosis. Usually, the sample's marker concentration is directly or indirectly correlated with a diagnosis and the marker concentration is e.g. used to determine whether an individual is at risk for a certain suffering from that disease. Alternatively the marker concentration can be compared to marker concentrations obtained from the same subject at an earlier time point. Alternatively, the sample's marker concentration can e.g., be compared to a marker concentration known to be associated with a response to therapy in a certain disease, the diagnosis of a certain disease, the assessment of the severity of a certain disease, the guidance for selecting an appropriate drug to a certain disease, in judging the risk of disease progression, or in the follow-up of patients. Depending on the intended diagnostic use an appropriate control sample is chosen and a control or reference value for the marker established therein. As also clear to the skilled artisan, the absolute marker values established in a control sample will be dependent on the assay used.

The most common control samples and/or reference values derived therefrom for the methods described herein are obtained from but not limited to “non-pathological controls” and “symptomatic controls”. The corresponding subjects from which these samples are obtained are “non-pathological subjects” and “symptomatic subjects” respectively.

“Non-pathological controls” refer to control samples of subjects that do not suffer from endometriosis and do not show any symptoms that could be associated with endometriosis (e.g., menstrual/abdominal pain, uterine/ovarian cysts or cancer, etc).

“Symptomatic controls” refer to control samples of subjects that suffer from symptoms that usually associated with endometriosis (e.g., menstrual/abdominal pain, infertility, etc) but where, based on laparoscopy, endometriosis can be excluded and no tissue alterations (e.g., uterine/ovarian cysts or cancer) can be observed.

“Controls with benign findings” refer to a group of samples of subjects that have tissue alterations (e.g., uterine/ovarian cysts, cancer, fibroids) which however does not resemble endometriosis. Further, these subjects can be symptomatic (e.g., menstrual/abdominal pain, infertility, etc) or asymptotic, which can also change over time.

The control sample may be assayed at the same time, before or after, separately or simultaneously with the test sample. The control value that is used in the comparison with the test sample may be a value that is calculated as an average or median of more than one (e.g., two or more, five or more, ten or more, a group etc) of control samples. Alternatively, the control sample may be a sample that originated from (i.e., is a mix of) more than one (e.g., two or more, five or more, ten or more, a group etc) individual that is not suffering from endometriosis (or is a “symptomatic control”).

In one example, the control sample is therefore obtained from a control subject that does not have endometriosis (“non-pathological control”). In a further example, the control sample is obtained from a subject that is a “symptomatic control”.

For classification of the stage of endometriosis a comparison of the test sample with several different control samples can or has to be carried out in order to be able to allocate the result to a certain stage. For example, a comparison to a non-pathologic sample and to a stage II sample. Or a comparison to a non-pathologic sample, to a stage II sample and to a stage IV sample. This can also be combined with surgery to confirm or determine a certain stage of disease.

Alternatively, the level of biomarker (e.g., protein) in the biological fluid sample may be compared to a pre-determined reference level for the biomarker of interest. As used herein, a “predetermined reference level” refers to a biomarker level obtained from a reference database, which may be used to generate a pre-determined cut off value, i.e., a score that is statistically predictive of endometriosis. In one example, the predetermined reference level is the average or median level of the biomarker in at least one individual not suffering from endometriosis from the same species. The predetermined reference value may be calculated as the average or median, taken from a group or population of individuals that are not suffering from endometriosis. For example, the predetermined reference value may be calculated as the average or median, taken from a group or population of individuals that are “symptomatic controls”. The individual or the population of individuals can be the same age or in the same state or condition of health as the subject from which the test sample is obtained.

In one example, the pre-determined reference level is therefore the average level of the biomarker in a control subject that does not have endometriosis. In a further example the pre-determined reference level is the average level of the biomarker in a subject that is a “symptomatic control”.

Typically, in methods for diagnosing endometriosis in a subject, the control sample or predetermined reference are obtained from an individual or group of individuals that are distinct from the subject that is being tested (i.e., the subject from which the test sample is obtained/provided). In such examples, the control or predetermined reference are used as a bench line to determine whether the tested subject has endometriosis.

In an alternative example, the control or predetermined reference value may be obtained from the same individual as the test sample, but at an earlier time point. This is particularly relevant for the methods described herein that classify the stage of endometriosis, that determine the progression in a subject, that determine the therapeutic effect of a treatment regimen for endometriosis, and/or that determine a subject's compliance or adherence with a prescribed treatment regimen for endometriosis. For this the samples are taken from the same biological fluid of the same subject, wherein the biological fluid is blood, serum, plasma, capillary blood, interstitial fluid, peritoneal fluid, or menstrual fluid preferably the biological fluid sample is serum.

In such examples, the control sample or predetermined reference level is used to determine any changes in the level of the biomarker(s) over a time interval for the same subject. The pre-determined reference level or control sample can therefore be from the same subject that the test sample is obtained from, for example obtained at an earlier time point. This earlier time point can be before they were diagnosed with endometriosis.

A pre-determined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of a biomarker of a population of individuals comprising the subject to be investigated. How to calculate a suitable reference value, preferably, the average or median, is well known in the art.

Thus, in some cases the level of the protein biomarker in a subject being greater than or equal to the level of the biomarker of the control sample or pre-determined reference level is indicative of a clinical status (e.g., indicative of endometriosis). In other cases, the level of the biomarker in a subject being less than or equal to the level of biomarker of the control sample or predetermined reference level is indicative of a certain stage of endometriosis.

Typically, but not necessarily, the greater than, or the less than, that is sufficient to distinguish a subject from a control subject is a statistically significantly greater than, or a statistically significant less than. In cases where the level of the biomarker in a subject being equal to the level of the biomarker in a control subject is indicative of a stage of endometriosis, the “being equal” refers to being approximately equal (e.g., not statistically different).

The pre-determined value can depend upon a particular population of subjects (e.g., human subjects) selected. For example, an apparently healthy population will have a different ‘normal’ range of the protein biomarker than will a population of subjects which have, or are likely to have, endometriosis. Accordingly, the pre-determined values selected may take into account the category (e.g., healthy, diseased, stage of disease) in which a subject (e.g., human subject) falls.

Appropriate ranges and categories can be selected with no more than routine experimentation by those of ordinary skill in the art.

Suitably, the level of the specific biomarker detected in a sample (e.g., a test sample, a control sample etc) may be normalized by adjusting the measured level (amount or activity) of the biomarker using the level of a reference protein in the same sample, wherein the reference protein is not a marker itself (it is e.g., a protein that is constitutively expressed). This normalization allows the comparison of the biomarker level in one sample to another sample, or between samples from different sources. This normalized level can then optionally be compared to a reference value or control. For example, when measuring a protein biomarker in a whole blood sample the biomarker may be expressed as an absolute concentration or, alternatively, it may be normalized against a known protein constitutively expressed in whole blood such as albumin, immunoglobulins or plasma protein concentration.

For example, when measuring a protein biomarker in a serum (or plasma) sample the biomarker may be expressed as an absolute concentration or, alternatively, it may be normalized against a known protein constitutively expressed in serum (or plasma).

The biomarker level(s) in the test sample may be compared to the level of the same biomarker in a control sample or with a pre-determined reference level for the same biomarker to identify an increase or decrease in a level of the one or more biomarker in the sample of the subject.

In the methods described herein, the subject may be identified as having endometriosis if the comparison (between biomarker level(s) in the control sample/predetermined reference value and the test sample of the subject) indicates that the subject has an increased level of c-Kit compared to the control sample or the pre-determined reference level.

The term “classifying” as used herein refers to the classification of the stage of endometriosis according to the revised scoring system of the American Society for Reproductive Medicine (r-ASRM) consisting of the four stages I, II, III and IV (Revised American Society for Reproductive Medicine classification of endometriosis: 1996. Fertil Steril. 1997). As shown in the data provided herein c-Kit levels at stages I, III and IV are about the same and are increased compared to the controls. c-Kit levels at stage II are increased compared to stages I, III and IV. This allows to classify, based on the comparison of the investigated sample with a reference value or with at least one sample investigated at an earlier stage, the status of endometriosis as being in accordance with the criteria to fall under one of the four stages based on the level of determined c-Kit. Depending on the reference value/reference sample differences between the levels of c-Kit in the sample of interest and the reference sample can be used to allocate the stage of endometriosis to a certain stage according to the r-ASRM. For example, if the c-Kit reference value is derived from a sample of a patient having endometriosis in stage II a decreased c-Kit level in the sample of interest would mean that the patient either has endometriosis at stage I, III or IV or that the patient does not have endometriosis. This depends on a comparison of the obtained c-Kit value with a reference value of an individual not suffering from endometriosis. Or in case the reference value is derived from a sample of a patient having endometriosis at stage I, III, or IV and increased level in the sample of interest would mean that the patient has stage II endometriosis. A classification can also be made if ranges for c-Kit levels are defined that are allocated to the certain stages of endometriosis. By this an obtained value falling within the range allocated to, for example, stage I would mean that this patient suffers from endometriosis stage I.

Moreover, it will be understood that if the risk of the deterioration of the health condition is predicted, typically, the prediction is made within a predictive window of 6 month and two years. More typically, said predictive window is about a time window of about 6 months to 12 month for a non-invasive test dependent on the symptoms, such as pelvic pain.

As will be understood by those skilled in the art, the assessment made in accordance with the present invention, although preferred to be, may usually not be correct for 100% of the investigated subjects. The term, typically, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc., Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The terms “lowered” or “decreased” level of an indicator refer to the level of such indicator in the sample being reduced in comparison to the reference (value) or reference sample. The terms “decrease”, “decreased” “reduced”, “reduction” or “down-regulated”, “lower” are all used herein generally to mean a decrease by a statistically significant amount. However, for avoidance of doubt, “reduced”, “reduction”, “decreased” or “decrease” means a decrease by at least 10% as compared to a reference level/control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference/control sample), or any decrease between 10-100% as compared to a reference level/control, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a reference level/control.

The terms “elevated” or “increased” level of an indicator/(bio) marker refer to the level of such indicator in the sample being higher in comparison to the reference (value) or reference sample. E.g. a protein that is detectable in higher amounts in a fluid sample of one individual suffering from a given disease than in the same fluid sample of individuals not suffering from said disease, has an elevated level. The terms “increased”, “increase” or “up-regulated”, “higher” are all used herein to generally mean an increase by a statically significant amount; for the avoidance of any doubt, the terms “increased” or “increase” means an increase of at least 10% as compared to a reference level/control, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level/control, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level/control.

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: a, y, d, E, and u. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CHI. CH2. CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCyl-Cy2-Cy3), whereas the human IgG light chain is composed of two immunoglobulin domains linked from N- to C-terminus in the order VL-CL, being either of the kappa or lambda type (VK-CK or VA.-CA.). Exemplified, the constant chain of human IgG comprises 447 amino acids. Throughout the present specification and claims, the numbering of the amino acid positions in an immunoglobulin are that of the “EU index” as in Kabat, E. A., Wu, T.T., Perry, H. M., Gottesman, K. S., and Foeller, C., (1991) Sequences of proteins of immunological interest, 5^{th}ed. U.S. Department of Health and Human Service, National Institutes of Health, Bethesda, MD. The “EU index as in Kabat” refers to the residue numbering of the human IgG IEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The terms “full-length antibody”, “intact antibody”, and “whole antibody” are used herein interchangeably to refer to an antibody in its substantially intact form, not antibody fragments as defined below. The terms particularly refer to an antibody with heavy chains that contain an Fc region.

Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFVB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually, linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

Accordingly, the term “antibody fragments” refers to a portion of an intact antibody, preferably comprising the antigen-binding region thereof. Antibody fragments include but are not limited to Fab, Fab′, F(ab′)2, Fv fragments; diabodies; sdAb, nanobodies, scFv, di-scFvs, tandem scFvs, triabodies, diabodies, scDb, BiTEs, and DARTs.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance-based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

“Sandwich immunoassays” are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g., an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 37° C. inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

An extremely versatile alternative sandwich assay format includes the use of a solid phase coated with the first partner of a binding pair, e.g., paramagnetic streptavidin-coated microparticles. Such microparticles are mixed and incubated with an analyte-specific binding agent bound to the second partner of the binding pair (e.g., a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay may include one or more washing step(s).

The term “detectably labeled” encompasses labels that can be directly or indirectly detected.

Directly detectable labels either provide a detectable signal or they interact with a second label to modify the detectable signal provided by the first or second label, e.g., to give FRET (fluorescence resonance energy transfer). Labels such as fluorescent dyes and luminescent (including chemiluminescent and electrochemiluminescent) dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling. In one embodiment detectably labeled refers to a label providing or inducible to provide a detectable signal, i.e., to a fluorescent label, to a luminescent label (e.g., a chemiluminescent label or an electrochemiluminescent label), a radioactive label or a metal-chelate based label, respectively.

Numerous labels (also referred to as dyes) are available which can be generally grouped into the following categories, all of them together and each of them representing embodiments according the present disclosure:

### (a) Fluorescent Dyes

Fluorescent dyes are e.g., described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oregon, USA) and Pierce Biotechnology, Inc. (Rockford, Ill.).

### (b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)3]2+ (which releases a photon at ˜620 nm) regenerating with TPA (Tripropylamine) in liquid phase or liquid-solid interface are used as ECL-labels.

Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescense is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605, 5,591,581, 5,597,910, PCT published application WO90/05296, PCT published application WO92/14139, PCT published application WO90/05301, PCT published application WO96/24690, PCT published application US95/03190, PCT application US97/16942, PCT published application US96/06763, PCT published application WO95/08644, PCT published application WO96/06946, PCT published application WO96/33411, PCT published application WO87/06706, PCT published application WO96/39534, PCT published application WO96/41175, PCT published application WO96/40978, PCT/US97/03653 and U.S. patent application Ser. No. 08/437,348 (U.S. Pat. No. 5,679,519). Reference is also made to a 1994 review of the analytical applications of ECL by Knight, et al. (Analyst, 1994, 119:879-890) and the references cited therein. In one embodiment the method according to the present description is practiced using an electrochemiluminescent label.

Recently also Iridium-based ECL-labels have been described (WO2012107419).

(c) Radioactive labels make use of radioisotopes (radionuclides), such as 3H, 11C, 14C, 18F, 32P, 35S, 64Cu, 68Gn, 86Y, 89Zr, 99TC, 111 In, 123I, 124I, 125I, 131I, 133Xe, 177Lu, 211At, or 131Bi.

(d) Metal-chelate complexes suitable as labels for imaging and therapeutic purposes are well-known in the art (US 2010/0111861; U.S. Pat. Nos. 5,342,606; 5,428,155; 5,316,757; 5,480,990; 5,462,725; 5,428,139; 5,385,893; 5,739,294; 5,750,660; 5,834,461; Hnatowich et al, J.

Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

The methods described herein can further comprise selecting, and optionally administering, a treatment regimen for the subject based on the diagnosis (i.e., based on the comparison of the levels of the biomarkers with the reference values/levels/controls). Treatment can include, for example, surgery and, in some cases, therapy, or combinations thereof. However, in some cases, immediate treatment may not be required, and the subject may be selected for active surveillance.

As used herein, the terms “active surveillance”, “monitoring” and “watchful waiting” are used interchangeably herein to mean closely monitoring a patient's condition without giving any treatment until symptoms appear or change.

As used herein, the terms “treat”, “treating” and “treatment” are taken to include an intervention performed with the intention of altering the pathology of a condition, disorder or symptom (i.e., in this case endometriosis). Accordingly, “treatment” refers to therapeutic treatment, wherein the object is to slow down (lessen) the targeted condition, disorder or symptom. “Treatment” therefore encompasses a reduction, slowing or inhibition of the symptoms of endometriosis, for example of at least 5%, 10%, 20%, 30%, 40%, 50%, 60%, 70%, 80%, 90% or 100% when compared to the symptoms before treatment. In the context of endometriosis, appropriate treatment may include pain medication, hormone treatments (such as hormonal contraceptives), gonadotropin-releasing hormone (GnRH) agonists, and/or surgery. (Longo, D. L et al. 2020. Sc. D. N Engl J Med, 382, pp. 1244-56).

As used herein, the term “surgery” applies to surgical methods undertaken for removal of endometric tissue, like, for example, laparoscopy or nerve sparing surgery.

As used herein, the term “therapy” includes drug-based therapy, radiation, hormonal therapy, cryosurgery, chemotherapy, immunotherapy, biologic therapy, and high-intensity focused ultrasound. Drug-based therapy of endometriosis can for example be by inhibiting or targeting neurogenic inflammation and/or pain medication and/or hormonal therapy.

The type of treatment will vary depending on the particular form and/or stage of endometriosis that the subject has, is suspected of having.

### Embodiments

The inventors have surprisingly identified the new protein biomarker c-Kit that is increased in biological fluids, in particular serum, of women with endometriosis, especially in women with early stages of endometriosis.

The biomarker c-Kit can be used for diagnosing endometriosis or classifying the stage of endometriosis in a subject compared to a control (e.g., non-pathological subjects or symptomatic subjects).

In particular, serum c-Kit can be used as a blood biomarker for early diagnosis and risk stratification of endometriosis. Furthermore, serum c-Kit can be used to select patients with disease stage I and stage II for early medical management of endometriosis. Therefore, it can significantly reduce endometriosis diagnostic delay, improve patients' lives, and reduce the economic burden.

The biomarker can advantageously be used in any of the methods, kits, assays, or uses described herein.

Methods for diagnosing endometriosis in a subject

In a first aspect the present invention relates to an in vitro method for diagnosing endometriosis in a subject, the method comprising the steps of:
a) determining the level of c-Kit in a biological fluid sample from the subject,
b) comparing the level of c-Kit to at least one appropriate reference value of a c-Kit level,
c) identifying a subject as having endometriosis if the comparison in step b) indicates that the subject has an increased level of c-Kit compared to the appropriate reference value.

In embodiments, an elevated level or amount or concentration of c-Kit in the fluid sample of the subject is indicative of the presence of endometriosis in the subject. In particular, an amount or concentration of c-Kit in the fluid sample of the subject is indicative of the presence of endometriosis in the subject if the amount or concentration of c-Kit in the fluid sample of the subject is higher than the amount or concentration of c-Kit according to a reference value.

In a particular embodiment the at least one appropriate reference value is
i. a level of c-Kit in a non-pathological subject or in a symptomatic subject, or is
ii. an average level of c-Kit in a group of non-pathological subjects or in a group of symptomatic subjects, or a combination thereof, or wherein the at least one appropriate reference value is
iii. a predetermined value of a level of c-Kit in a non-pathological subject or symptomatic subject, or is
iv. a predetermined average value of a level of c-Kit in a group of non-pathological subjects or in a group of symptomatic subjects, or a combination thereof.

In a particular embodiment, the inventors could detect an increase of serum c-Kit in the early stages of endometriosis, followed by a decrease in the late stages of endometriosis. However, the amount or concentration of c-Kit at late stages is still higher compared to the control levels.

In particular, an amount of c-Kit elevated by 50% or more, is indicative of the presence or the risk of developing of endometrioses. In particular, an amount of c-Kit elevated by 100% or more, is indicative of the presence of endometrioses. In particular, an amount of c-Kit elevated by 150% or more, is indicative of the presence of endometrioses. In particular, an amount of c-Kit elevated by 200% or more, is indicative of endometrioses.

Suitably, the biological fluid sample is blood, serum, plasma, capillary blood, interstitial fluid, peritoneal fluid, or menstrual fluid, preferably the biological fluid sample is serum.

In embodiments, the sample is an in vitro sample, i.e., it will be analyzed in vitro and not transferred back into the body of the subject. In embodiments, the method of the present invention is an in vitro method.

In particular embodiments, the subject is a human subject. In particular embodiments, the patient is a female human subject. In particular embodiments, the subject is a young or adolescent human female. In particular, the subject is a subject subject who is capable of suffering from endometriosis due to the physical condition.

In embodiments, the diagnosis is performed independent of the rASRM staging. In particular, the assessment is carried out without performing laparoscopy. In particular, the assessment is performed without assessing the presence or severity of endometriosis in the patient using laparoscopy and/or the rASRM staging.

In embodiments, the endometriosis diagnosed is selected from the group consisting of stage I endometriosis according to rASRM staging, stage II endometriosis according to rASRM staging, stage III endometriosis according to rASRM staging, stage IV endometriosis according to rASRM staging. In particular embodiments, the endometriosis diagnosed is stage I, stage II, stage III, or stage IV endometriosis.

In embodiments, endometriosis is early endometriosis, in particular stage I endometriosis according to rASRM staging or stage II endometriosis according to rASRM staging.

In particular embodiments, the endometriosis diagnosed is stage III or stage IV endometriosis.

In embodiments, the endometriosis assessed is selected from the group consisting of peritoneal endometriosis, endometrioma, deep infiltrating endometriosis (DIE), and adenomyosis.

In particular embodiments, the endometriosis diagnosed is peritoneal endometriosis. In other particular embodiments the endometriosis diagnosed is peritoneal endometriosis of stage I or stage II according to rASRM staging.

In another embodiment the methods further comprise selecting a treatment regimen for the subject based on the comparison of the level of c-Kit with the control sample or with the pre-determined reference level. In particular embodiments, the method further comprises administering the selected treatment regimen to the subject, optionally wherein the selected treatment regimen comprises drug-based therapy and/or surgical treatment (laparoscopy). Drug-based therapy of endometriosis can for example be by pain medication hormone treatments, and/or surgery.

Depending on whether the diagnosis rather hints to a severe disease stage or not the person skilled in the art would be well aware of how to select the most appropriate and promising treatment regimen.

In embodiments according to the invention, the protein level of c-Kit is determined, optionally using a process selected from: ELISA assay, immunoblotting, lateral flow assay, protein microarray and mass spectrometry.

In embodiments, the amount of c-Kit is determined using antibodies, in particular using monoclonal antibodies. In embodiments, step a) of determining the amount of c-Kit in a sample of the patient comprises performing an immunoassay. In embodiments, the immunoassay is performed either in a direct or indirect format. In embodiments such immunoassays are selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In particular embodiments, step a) of determining the level of c-Kit in a sample of the subject comprises the steps of
i) incubating the sample of the subject with one or more antibodies specifically binding to c-Kit, thereby generating a complex between the antibody and c-Kit, and
ii) quantifying the complex formed in step i), thereby quantifying the amount of c-Kit in the sample of the subject.

In particular embodiments, in step i) the sample is incubated with two antibodies, specifically binding to c-Kit. As obvious to the skilled artisan, the sample can be contacted with the first and the second antibody in any desired order, i.e., first antibody first and then the second antibody or second antibody first and then the first antibody, or simultaneously, for a time and under conditions sufficient to form a first anti-c-Kit antibody/c-Kit/second anti-c-Kit antibody complex. As the skilled artisan will readily appreciate it is nothing but routine experimentation to establish the time and conditions that are appropriate or that are sufficient for the formation of a complex either between the specific anti-c-Kit antibody and the c-Kit antigen/analyte (=anti-c-Kit complex) or the formation of the secondary, or sandwich complex comprising the first antibody to c-Kit, c-Kit (the analyte) and the second anti-c-Kit antibody (=anti-c-Kit antibody/c-Kit/second anti-c-Kit antibody complex).

The detection of the anti-c-Kit antibody/c-Kit complex can be performed by any appropriate means. The detection of the first anti-c-Kit antibody/c-Kit/second anti-c-Kit antibody complex can be performed by any appropriate means. The person skilled in the art is absolutely familiar with such means/methods.

In certain embodiments a sandwich will be formed comprising a first antibody to c-Kit, c-Kit (analyte) and the second antibody to c-Kit, wherein the second antibody is detectably labeled.

In one embodiment a sandwich will be formed comprising a first antibody to c-Kit, the c-Kit (analyte) and the second antibody to c-Kit, wherein the second antibody is detectably labeled and wherein the first anti-c-Kit antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In embodiments, the method further comprising the assessment of dysmenorrhea and/or lower abdominal pain in the patient. In embodiments the presence of dysmenorrhea and/or lower abdominal pain is assessed according to the VAS scale. In embodiments, dysmenorrhea VAS score of 4 or higher indicated moderate or severe dysmenorrhea. In embodiments, scores of 3 or less indicate no or mild dysmenorrhea.

In embodiments, the method further comprising determining the level of CA-125 in the biological fluid sample from the subject.

Methods for classifying the stage of endometriosis in a subject

In a second aspect the invention relates to an in vitro method for classifying the stage of endometriosis in a subject, the method comprising the steps of:
a) determining the level of c-Kit in a biological fluid sample from the subject,
b) comparing the level of c-Kit to at least one appropriate reference value of a c-Kit level,
c) classifying the stage of endometriosis in the subject if the comparison in step b) indicates that the subject has an increased or decreased level of c-Kit compared to the at least one appropriate reference value of a c-Kit level.

In embodiments, an elevated or reduced level or amount or concentration of c-Kit in the fluid sample of the subject can be indicative of the stage of endometriosis in the subject. In particular, a level of c-Kit in the fluid sample of the subject is indicative of the stage of endometriosis in the subject if the level of c-Kit in the fluid sample of the subject is higher or lower than the level of c-Kit according to a reference value of which the stage of endometriosis is known. If necessary, comparisons to several reference values are made in order to classify the stage of endometriosis. In addition this can be combined with surgery or other parameters used to determine the stage of endometriosis to allow an even more precise classification.

In particular, if c-Kit is detectable in higher levels in a fluid sample of the subject assessed for the presence of endometriosis than in the same fluid sample of subjects not suffering from endometriosis then this is indicative of any stage of endometriosis, A slight increase would be indicative for stages I, III and IV, whereas a stronger increase would be indicative for stage II. By further comparing the obtained value to reference values of which the stage of endometriosis is known an allocation of the obtained value to a certain stage of endometriosis is possible.

In a particular embodiment the at least one appropriate reference value is
i. a level of c-Kit in a non-pathological subject, in a symptomatic subject, or in a subject that has stage I, stage II, stage III or stage IV endometriosis according to the revised scoring system of the American Society for Reproductive Medicine (r-ASRM), or is
ii. an average level of c-Kit in a group of subjects that are non-pathological subjects, that are symptomatic subjects, or a combination thereof, or in a group of subjects that have stage I, stage II, stage III or stage IV endometriosis according to the revised scoring system of the American Society for Reproductive Medicine (r-ASRM),

or wherein the at least one appropriate reference value is
iii. a predetermined value of a level of c-Kit in a non-pathological subject or in a symptomatic subject, or in a subject that has stage I, stage II, stage III or stage IV endometriosis according to the revised scoring system of the American Society for Reproductive Medicine (r-ASRM), or is
iv. a predetermined average value of a level of c-Kit in a group of non-pathological subjects, that that are symptomatic subjects, or a combination thereof, or in a group of subjects that have stage I, stage II, stage III or stage IV endometriosis according to the revised scoring system of the American Society for Reproductive Medicine (r-ASRM).

In a particular embodiment, the inventors could detect an increase of serum c-Kit in the early stages of endometriosis, followed by a decrease in the late stages of endometriosis. However, the amount or concentration of c-Kit at late stages is still higher compared to the control levels.

For example, a level of c-Kit obtained that is higher than in a non-pathological subject but lower than in a subject with stage II endometriosis is indicative of stage I, III, or IV endometriosis.

For example, a level of c-Kit obtained that is higher than in a subject with stage I endometriosis and higher than in a subject with stage III endometriosis is indicative of stage II endometriosis.

Details of the biomarkers, combinations, samples, methods steps, subjects, types of endometriosis, treatment, reference values, etc are provided elsewhere and apply equally to this and all the other aspects.

### Methods for Monitoring Endometriosis Progression in a Subject

In a third aspect the present invention relates to an in vitro method for monitoring endometriosis progression in a subject, the method comprising the steps of:
i. determining the level of c-Kit in a biological fluid sample from the subject in accordance with method steps a) to b) described above herein,
ii. repeating step i. using a biological fluid sample obtained from the subject during or after treatment for a time interval; and
iii. comparing the levels of c-Kit identified in i. with the c-Kit levels identified in ii., wherein a change in the c-Kit levels from i. to ii. is indicative of a change in endometriosis progression in the subject.

In embodiments, a patient suffering from endometriosis is monitored to determine if the amount or concentration of c-Kit is changing over time in a sample of the patient. In particular, a patient suffering from endometriosis is monitored to determine if the amount or concentration of c-Kit is increasing, decreasing or not changing over time. In embodiments, a patient suffering from endometriosis is monitored if an elevated amount of c-Kit in the sample of the patient is determined.

The method may be used to monitor the progression of any kind of endometriosis described herein.

Typically, such monitoring methods are performed on subjects that have not yet been treated for endometriosis (i.e., they have not previously received endometriosis treatment (therapy or surgery)). Such subjects are described as “naïve” subjects herein.

However, such monitoring methods also encompass methods performed on subjects that have already been treated for endometriosis.

Monitoring the progression of endometriosis in a subject over time assists in the earliest possible identification of disease progression (e.g., a worsening in disease status or disease symptoms). Such monitoring naturally involves the taking of repeated samples over time. The method may therefore be repeated at one or more time intervals for a particular subject and the results compared to monitor the development, progression or improvement in endometriosis of that subject over time, wherein a change in the amount of level of the biomarker tested for in the biological fluid sample (e.g., serum) is indicative of a change in the progression of the endometriosis in the subject.

Some studies have reported that serum CA-125 levels were reduced after surgery of endometriosis followed by medical treatment (Chen et al. 1998. Acta Obstet Gynecol Scand. 77:665-70, Jacobs et al. 1989. Hum Reprod. 4:1-12).

Disease progression may be indicated by an increase in the level of c-Kit detected over time when the results of two or more time intervals are compared for the same subject.

In other words, if the method is performed a plurality of times, disease progression may be indicated when the level of c-Kit detected at the later time interval(s) is higher than that detected at the earlier time interval(s). An “increase” in the level of c-Kit encompasses detection of c-Kit at a later time interval when no c-Kit was detected (i.e., it was not present at detectable levels) when the method was performed previously (i.e., at an earlier time interval) on the same subject (and an equivalent biological fluid sample type). This is particularly relevant when monitoring the progression of endometriosis in naïve subjects.

Suitable time intervals for monitoring disease progression can easily be identified by a person of skill in the art and will depend on the specific form of endometriosis being monitored. As a non-limiting example, the method may be repeated at least every week, month, six months, or at least every year, or whenever clinically needed, i.e., in case of a significant change in endometriosis symptoms.

Details of the biomarkers, combinations, samples, methods steps, subjects, types of endometriosis, treatments, reference values, etc are provided elsewhere and apply equally to this and all the other aspects.

Methods for determining the therapeutic effect of a treatment regimen for endometriosis in a subject

In a fourth aspect the present invention relates to an in vitro method for determining the therapeutic effect of a treatment regimen for endometriosis in a subject, the method comprising the steps of:
i. determining the level of c-Kit in a biological fluid sample from the subject in accordance with method steps a) to b) described above herein,
ii. repeating step a) using a biological fluid sample obtained from the subject during or after treatment for a time interval; and
iii. comparing the level of c-Kit determined in step a) to that determined in step b), and identifying that the treatment regimen has a therapeutic effect if the level of c-Kit decreased after treatment.

In one example, the change in level of c-Kit that is indicative of a therapeutic effect is a decrease in c-Kit level after treatment. An “decrease” in the level of c-Kit encompasses no detection of c-Kit (i.e., it is not present at detectable levels) at a later time interval when c-Kit was detected when the method was performed previously (i.e. at an earlier time interval) on the same subject (and an equivalent biological fluid sample type).

Step i. may first be performed in accordance with the method using a biological fluid sample that was obtained from the subject at a time point before the treatment regimen for endometriosis began. Alternatively, step i. may first be performed using a biological fluid sample that was obtained from the subject at the same time as commencing the treatment regimen, or at a time point after the treatment regimen for endometriosis began. The method can therefore be used to determine the therapeutic effect of a treatment regimen for endometriosis from the outset (i.e., from the start of the regimen) or from a time point after the treatment regimen has started (i.e., determining the therapeutic effect of a treatment regimen for endometriosis during the treatment regimen itself).

In embodiments, an unaltered or increasing amount or concentration of c-Kit in a sample of the subject being treated for endometriosis is indicative of the therapy being ineffective, i.e., an unaltered or increasing amount or concentration of c-Kit in a sample of the subject being treated for endometriosis is indicative of persisting or recurring endometriosis. In particular, the treatment for endometriosis is ineffective if the amount of c-Kit is increasing to 50% or more. In particular, the treatment for endometriosis is ineffective if the amount of c-Kit is increasing to 100% or more. In particular, the treatment for endometriosis is ineffective if the amount of c-Kit is increasing to 150% or more. In particular, the treatment for endometriosis is ineffective if the amount of c-Kit is increasing to 200% or more.

Alternatively, an unaltered level of c-Kit could either mean that the disease stagnates or that it has been progressed from stage I to stages III or IV where the c-Kit levels are comparable to stage I.

An improvement in disease status or symptoms (e.g., over a treatment period) may also be indicated by stabilised levels of c-Kit over time (compared to the level of c-Kit observed in the absence of treatment over the equivalent time period or compared to equivalent controls).

A treatment regimen may be identified as having a therapeutic effect if it results in a delay in disease progression or a delay in the development of symptoms (e.g., over a treatment period).

A treatment regimen may also be identified as having a therapeutic effect if it results in an improvement in disease status or symptoms (e.g., over a treatment period). Methods for determining if the treatment regimen has a therapeutic effect are well known in the art.

A treatment period refers to a time interval over which treatment occurs (e.g., 1 month, 3 months, 6 months, 1 year, 2 years, etc).

As would be clear to a person of skill in the art, the direction of change in c-Kit levels that is indicative of a therapeutic effect may depend on the disease status of the subject prior to treatment and the control/reference used.

The change in level of c-Kit can also be indicative of compliance or adherence with the prescribed treatment after treatment.

The trends for identifying that the subject has complied or adhered with the prescribed treatment regimen are equivalent to those described in detail above in respect of determining the therapeutic effect of a treatment regimen for endometriosis. This is because a “prescribed treatment regimen” is a recommended treatment regimen and therefore typically has a therapeutic effect (and thus, observation of the therapeutic effect on the biomarker levels is an indication of subject compliance or adherence with the prescribed treatment regimen).

In embodiments, the subject is monitored several times at different time points. In embodiments, the patient is monitored several times within a time frame of weeks, months, or years. In particular embodiments, a subject is monitored is once a month or once a year. In embodiments, a subject suffering from endometriosis is monitored once a month or once a year after diagnosis of endometriosis. In embodiments, a subject being treated for endometriosis is monitored once after therapy, in particular once after surgical therapy. In particular, the subject being treated for endometriosis is monitored once a month or once a year to determine the efficacy of treatment and/or the recurrence of endometriosis.

The method can also be useful as a screening tool for determining if specific regimens or treatment modalities have a therapeutic effect on endometriosis. The tested regimens or treatment modalities may be new regimens or treatment modalities, modified regimens or treatment modalities, or known regimens or treatment modalities that need further testing. In this context, a treatment modality is e.g., a drug or medicament that is useful or suspected to be useful in the treatment of endometriosis.

In embodiments, therapy of endometriosis is selected from the group consisting of drug-based therapy or surgical therapy. In embodiments the treatment regimen comprises surgical therapy, radiotherapy, immunotherapy, hormone therapy, ultrasound therapy, or combinations thereof. In preferred embodiments, surgical therapy of endometriosis is laparoscopy or nerve sparing surgery. In embodiments, drug-based therapy of endometriosis is inhibiting or targeting neurogenic inflammation and/or pain medication and/or hormonal therapy.

In particular embodiments, therapy is adapted if an unaltered or increasing amount or concentration of c-Kit in a sample of the patient being treated for endometriosis is determined.

Details of the biomarkers, combinations, samples, methods steps, subjects, types of endometriosis, treatments, etc are provided elsewhere and apply equally to this aspect.

Accordingly, all aspects described in detail above for methods for determining the therapeutic effect of a treatment regimen for endometriosis apply equally here.

Details of the biomarkers, combinations, samples, methods steps, subjects, types of endometriosis, treatments, reference values, etc are provided elsewhere and apply equally to this and all the other aspects.

Computer-Implemented Method for Assessing a Patient with Endometriosis

In a fourth aspect the present invention relates to a computer-implemented method for assessing a patient with suspected endometriosis comprising the steps of:
(a) receiving a value for level of a first biomarker in a sample of the subject, said first biomarker being c-Kit,
(b) receiving a value for the level of a second biomarker in a sample of the subject, wherein said second biomarker is CA125,
(c) receiving a value for the level of dysmenorrhea according to the VAS and/or lower abdominal pain according to the VAS,
(d) comparing the values for the levels of steps (a)-(c) to references for said biomarkers and the amount of dysmenorrhea and/or calculating a score for assessing the subject with suspected endometriosis based on the levels of the biomarkers and the amount of dysmenorrhea; and
(e) assessing said subject based on the comparison and/or the calculation made in step (d).

The term “computer-implemented” as used herein means that the method is carried out in an automated fashion on a data processing unit which is, typically, comprised in a computer or similar data processing device. The data processing unit shall receive values for the amount of the biomarkers. Such values can be the amounts, relative amounts or any other calculated value reflecting the amount as described elsewhere herein in detail. Accordingly, it is to be understood that the aforementioned method does not require the determination of amounts for the biomarkers but rather uses values for already predetermined amounts.

The present invention also, in principle, contemplates a computer program, computer program product or computer readable storage medium having tangibly embedded said computer program, wherein the computer program comprises instructions which, when run on a data processing device or computer, carry out the method of the present invention as specified above.

Specifically, the present disclosure further encompasses:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a data for parameters as defined herein elsewhere, and
a data stream signal, typically encrypted, comprising the assessment provided by the methods of the present invention.
   Combination with CA-125, Symptoms or Clinical Data

The methods according to the present invention can be combined with other tests, biomarkers, clinical data or further information that is useful to diagnose or classify endometriosis in order to obtain the most reliable result.

Despite its rather weak diagnosis performance CA-125 is routinely used as a biomarker for endometriosis. Accordingly, it may be advantageous to combine determining the level of c-Kit with CA-125 obtained from the subject in the context of the methods described herein.

Besides CA-125 other symptoms or clinical data that is used for diagnosing or classifying endometriosis can be used in combination with the determination of c-Kit levels. Such symptoms or clinical data can be but are not limited to age, dysmenorrhea, abdominal pain, or other biomarkers.

### Kits and Devices

In another aspect, kits are provided for diagnosing or classifying the stage of endometriosis in a subject. The kits include reagents suitable for determining levels of a plurality of analytes in a test sample (e.g., reagents suitable for determining levels of the biomarker disclosed herein).

The kits described herein typically comprise a detectably labelled agent that specifically binds to c-Kit protein.

Such a kit may additionally comprise a detectably labelled agent that specifically binds to CA-125.

The kits described herein can take on a variety of forms. Typically, the kits will include reagents suitable for determining levels of a plurality of biomarkers (for example c-Kit and optionally CA-125) in a sample.

Optionally, the kits may contain one or more control samples or references. Typically, a comparison between the levels of the biomarkers in the subject and levels of the biomarkers in the control samples is indicative of a clinical status (e.g., diagnosis of endometriosis). Also, the kits, in some cases, will include written information (indicia) providing a reference (e.g., pre-determined values), wherein a comparison between the levels of the biomarker in the subject and the reference (pre-determined values) is indicative of a clinical status (e.g., diagnosis of endometriosis). In some cases, the kits comprise software useful for comparing biomarker levels or occurrences with a reference (e.g., a prediction model). Usually, the software will be provided in a computer readable format such as a compact disc, but it also may be available for downloading via the internet. However, the kits are not so limited and other variations with will be apparent to one of ordinary skill in the art.

The components of the kit may be housed in a container that is suitable for transportation. Details on the biomarker is given above and apply equally here. Suitably, the biomarker may be protein.

In some examples the kits include the detectably labelled agent(s) on a continuous (e.g., solid) surface, such as a lateral flow surface. Alternatively, in examples comprising more than one detectably labelled agent, the detectably labelled agent(s) may be located in distinct (i.e., spatially separate) zones on a (e.g., solid) surface, such as a multiwall micro-titre plate (e.g., for an ELISA assay). Other appropriate surfaces and containers that are well known in the art may also form part of the kits described herein.

In one example, the kit further comprises one or more reagents for detecting the detectably labelled agent. Suitable reagents are well known in the art and include but are not limited to standard reagents and buffers required to perform any one of the appropriate detection methods that may be used (and are well known in the art).

In one example, the kit comprises one or more of the following: a multi-well plate, ball bearing(s), extraction buffer, extraction bottle and a lateral flow device lateral flow device.

An assay device is also provided for diagnosing endometriosis in a subject.

Typically, the device comprises a surface with at least one detectably labelled agent located thereon that specifically binds to c-Kit protein.

Such a device may additionally comprise a detectably labelled agent that specifically binds to CA-125.

If two detectably labelled agents are used, they may be located in separate zones on the surface. In other words, the two detectably labelled agents may be located in distinct (i.e., spatially separate) zones on a (e.g., solid) surface, such as a multiwell micro-titre plate. Detectably labelled agent(s) that specifically bind to the biomarker(s) of interest are described in detail elsewhere herein.

The assay device comprises a surface upon which the detectably labelled agents are located. Appropriate surfaces include a continuous (e.g., solid) surface, such as a lateral flow surface, a dot blot surface, a dipstick surface or a surface suitable for performing surface plasmon resonance. Other appropriate surfaces include microtitre plates, multi-well plates etc. Other appropriate surfaces that are well known in the art may also form part of the assay device described herein.

Appropriate assay device formats therefore include but are not limited to device formats suitable for performing any one of lateral flow, dot blot, ELISA, or surface plasmon resonance assays for detecting the presence, level or absence of the biomarker of interest.

### Data Storage Aspects

Biomarker levels and/or reference levels may be stored in a suitable data storage medium (e.g., a database) and are, thus, also available for future diagnoses. This also allows efficiently diagnosing prevalence for a disease because suitable reference results can be identified in the database once it has been confirmed (in the future) that the subject from which the corresponding reference sample was obtained did have endometriosis. As used herein a “database” comprises data collected (e.g., analyte and/or reference level information and/or patient information) on a suitable storage medium. Moreover, the database, may further comprise a database management system. The database management system is, preferably, a network-based, hierarchical or object-oriented database management system. Furthermore, the database may be a federal or integrated database. More preferably, the database will be implemented as a distributed (federal) system, e.g., as a Client-Server-System. More preferably, the database is structured as to allow a search algorithm to compare a test data set with the data sets comprised by the data collection. Specifically, by using such an algorithm, the database can be searched for similar or identical data sets being indicative of endometriosis (e.g., a query search). Thus, if an identical or similar data set can be identified in the data collection, the test data set will be associated with endometriosis. Consequently, the information obtained from the data collection can be used to diagnose endometriosis or based on a test data set obtained from a subject. More preferably, the data collection comprises characteristic values of all analytes comprised by any one of the groups recited above.

The methods described herein may further include communication of the results or diagnoses (or both) to technicians, physicians or patients, for example. In certain examples, computers will be used to communicate results or diagnoses (or both) to interested parties, e.g., physicians and their patients.

In some examples, the results or diagnoses (or both) are communicated to the subject as soon as possible after the diagnosis is obtained. The results or diagnoses (or both) may be communicated to the subject by the subject's treating physician. Alternatively, the results or diagnoses (or both) may be sent to a subject by email or communicated to the subject by phone. A computer may be used to communicate the results or diagnoses by email or phone. In certain examples, the message containing results or diagnoses may be generated and delivered automatically to the subject using a combination of computer hardware and software which will be familiar to artisans skilled in telecommunications.

### Uses

Also provided herein is the use of the biomarker c-Kits as a biological fluid biomarker for endometriosis.

In a preferred example c-Kit may be used as biomarker for endometriosis generally. In this context “endometriosis generally” refers to all forms of endometriosis, including but not limited to peritoneal endometriosis, endometrioma, deep infiltrating endometriosis, and adenomyosis.

c-Kit may also be combined with CA-125.

Details of the biomarkers, samples, methods, subjects, types of endometriosis etc are provided elsewhere and apply equally to this aspect.

### Companion Diagnostic

The methods kits, assay devices and uses provided herein may be used as part of a companion diagnostic e.g., as part of a medical device, often an in vitro device, which provides information that is essential for the safe and effective use of a corresponding drug or biological product (wherein the corresponding drug or biological product is for treating or preventing endometriosis).

In further embodiments, the present invention relates to the following aspects:

1. An in vitro method for diagnosing endometriosis in a subject, the method comprising the steps of:
a) determining the level of c-Kit in a biological fluid sample from the subject,
b) comparing the level of c-Kit to at least one appropriate reference value of a c-Kit level,
c) identifying a subject as having endometriosis if the comparison in step b) indicates that the subject has an increased level of c-Kit compared to the appropriate reference value.

2. The method according to aspect 1, wherein the at least one appropriate reference value is
i. a level of c-Kit in a non-pathological subject or in a symptomatic subject, or is
ii. an average level of c-Kit in a group of non-pathological subjects or in a group of symptomatic subjects, or a combination thereof, or wherein the at least one appropriate reference value is
iii. a predetermined value of a level of c-Kit in a non-pathological subject or symptomatic subject, or is
iv. a predetermined average value of a level of c-Kit in a group of non-pathological subjects or in a group of symptomatic subjects, or a combination thereof.

3. An in vitro method for classifying the stage of endometriosis in a subject, the method comprising the steps of:
a) determining the level of c-Kit in a biological fluid sample from the subject,
b) comparing the level of c-Kit to at least one appropriate reference value of a c-Kit level,
c) classifying the stage of endometriosis in the subject if the comparison in step b) indicates that the subject has an increased or decreased level of c-Kit compared to the at least one appropriate reference value of a c-Kit level.

4. An in vitro method for classifying the stage of endometriosis in a subject, the method comprising the steps of:
a) determining the level of c-Kit in a biological fluid sample from the subject at regular intervals,
b) comparing the level of c-Kit to at least one value of a c-Kit level determined at an earlier stage in the subject,
c) classifying the stage of endometriosis in the subject if the comparison in step b) indicates that the subject has an increased or decreased level of c-Kit compared to the at least one value of a c-Kit level determined at an earlier stage in the subject.

5. The method according to aspect 3 or 4, wherein the at least one appropriate reference value is
i. a level of c-Kit in a non-pathological subject, in a symptomatic subject, or in a subject that has stage I, stage II, stage III or stage IV endometriosis according to the revised scoring system of the American Society for Reproductive Medicine (r-ASRM), or is
ii. an average level of c-Kit in a group of subjects that are non-pathological subjects, that are symptomatic subjects, or a combination thereof, or in a group of subjects that have stage I, stage II, stage III or stage IV endometriosis according to the revised scoring system of the American Society for Reproductive Medicine (r-ASRM),

or wherein the at least one appropriate reference value is
iii. a predetermined value of a level of c-Kit in a non-pathological subject or in a symptomatic subject, or in a subject that has stage I, stage II, stage III or stage IV endometriosis according to the revised scoring system of the American Society for Reproductive Medicine (r-ASRM), or is
iv. a predetermined average value of a level of c-Kit in a group of non-pathological subjects, that that are symptomatic subjects, or a combination thereof, or in a group of subjects that have stage I, stage II, stage III or stage IV endometriosis according to the revised scoring system of the American Society for Reproductive Medicine (r-ASRM).

6. The method according to any preceding aspect, wherein the biological fluid sample is blood, serum, plasma, capillary blood, interstitial fluid, peritoneal fluid, or menstrual fluid preferably the biological fluid sample is serum.

7. The method according to any preceding aspect, wherein the subject is a human, preferably a female human.

8. The method according to any preceding aspect, wherein the protein level of c-Kit is determined, optionally using a process selected from: ELISA assay, immunoblotting, lateral flow assay, protein microarray and mass spectrometry.

9. The method of any preceding aspects, further comprising administering a selected treatment regimen to the subject, optionally wherein the selected treatment regimen comprises surgery, radiotherapy, immunotherapy, hormone therapy, ultrasound therapy, or combinations thereof.

10. The method according to any preceding aspect, wherein the diagnosis or classification is performed independent of the scoring system of the American Society for Reproductive Medicine

11. The method according to any of aspects 3-10, wherein the subject's stage of endometriosis is classified as stage I, stage II, stage III or stage IV endometriosis according to the revised scoring system of the American Society for Reproductive Medicine (r-ASRM).

12. The method according to any of aspects 3-11, wherein the subject's stage of endometriosis is classified as stage I or stage II endometriosis according to the revised scoring system of the American Society for Reproductive Medicine (r-ASRM).

13. The method according to any preceding aspect, wherein endometriosis is selected from the group consisting of peritoneal endometriosis, endometrioma, deep infiltrating endometriosis, and adenomyosis.

14. The method of any preceding aspect, further comprising selecting a treatment regimen for the subject based on the comparison of the level of c-Kit with the control sample or with the pre-determined reference level.

15. The method according to aspect 14, further comprising administering the selected treatment regimen to the subject, optionally wherein the selected treatment regimen comprises drug-based therapy and/or surgical treatment (laparoscopy).

16. An in vitro method for monitoring endometriosis progression in a subject, the method comprising the steps of:
i. determining the level of c-Kit in a biological fluid sample from the subject in accordance with method steps a) to b) of any one of aspects 1 to 15,
ii. repeating step i. using a biological fluid sample obtained from the subject during or after treatment for a time interval; and
iii. comparing the levels of c-Kit identified in i. with the c-Kit levels identified in ii., wherein a change in the c-Kit levels from i. to ii. is indicative of a change in endometriosis progression in the subject.

17. An in vitro method for determining the therapeutic effect of a treatment regimen for endometriosis in a subject, the method comprising the steps of:
i. determining the level of c-Kit in a biological fluid sample from the subject in accordance with method steps a) to b) of any one of aspects 1 to 15,
ii. repeating step i. using a biological fluid sample obtained from the subject during or after treatment for a time interval; and
iii. comparing the level of c-Kit determined in step i. to that determined in step ii. and identifying that the treatment regimen has a therapeutic effect if the level of c-Kit decreased after treatment.

18. The method according to any preceding aspect, further comprising the assessment of dysmenorrhea according to the Visual Analog Scale (VAS) and/or lower abdominal pain according to VAS.

19. The method according to any preceding aspect, further comprising determining the level of CA-125 in the biological fluid sample from the subject.

20. The method according to aspect 18, comprising calculating
a ratio of the amount or concentration of c-Kit and the amount or concentration of CA-125, or
a ratio of the amount or concentration of c-Kit and dysmenorrhea, or
a ratio of the amount or concentration of c-Kit and the amount or concentration of CA-125 and dysmenorrhea, or
a ratio of the amount or concentration of c-Kit and lower abdominal pain according to the VAS scale.

21. A computer-implemented method for assessing a patient with suspected endometriosis comprising the steps of:
(a) receiving a value for level of a first biomarker in a sample of the subject, said first biomarker being c-Kit,
(b) receiving a value for the level of a second biomarker in a sample of the subject, wherein said second biomarker is CA125,
(c) receiving a value for the level of dysmenorrhea according to the VAS and/or lower abdominal pain according to the VAS,
(d) comparing the values for the levels of steps (a)-(c) to references for said biomarkers and the amount of dysmenorrhea and/or calculating a score for assessing the subject with suspected endometriosis based on the levels of the biomarkers and the amount of dysmenorrhea; and
(e) assessing said subject based on the comparison and/or the calculation made in step (d).

22. Use of elevated c-Kit levels in a biological fluid sample as a biomarker for endometriosis.

23. The use according to aspect 22, wherein the biological fluid sample is blood or blood-derived, preferably serum.

24. The use according to aspects 22 and 23, wherein the use is to diagnose and/or classify endometriosis.

25. A kit for diagnosing and/or classifying endometriosis in a subject, comprising at least one detectably labelled agent that specifically binds to c-Kit protein.

26. The kit according to aspect 25, further comprising one or more reagents for detecting the detectably labelled agent.

27. An assay device for diagnosing and/or classifying endometriosis in a subject, the device comprising a surface with at least one detectably labelled agent located thereon that specifically binds to c-Kit protein.

Aspects of the invention are demonstrated by the following non-limiting examples. The following examples and figures are provided to aid the understanding of the present invention, the true scope of which is set forth in the appended claims. It is understood that modifications can be made in the procedures set forth without departing from the spirit of the invention.

### EXAMPLES

### Example 1: Diagnostic Performance of Biomarker c-Kit in Women with Endometriosis

For the measurements, a total of 250 serum samples from human females were analysed (for clinical data it is referred to the corresponding section herein). The concentration of the analytes was determined by ELISA (enzyme-linked immunosorbent assay). The case group is comprised of patients diagnosed with endometriosis (peritoneal endometriosis, adenomyosis, endometrioma, and deep infiltrating endometriosis; rASRM stages I-IV) diagnosed by laparoscopic with subsequent histological confirmation and the control group including healthy women without endometriosis.

The concentration of c-Kit in human serum was determined using the Human CD117/c-Kit Quantikine ELISA Kit (R&D Systems, USA (catalogue number: DSCR00). The kit utilizes the quantitative sandwich ELISA technique. Microtiter plates are pre-coated with a monoclonal antibody specific for human c-Kit. Samples are measured in 50-fold dilution. After bringing all reagents to room temperature 100 μL of each sample and standard are added. Samples are measured in singlicates, standards in duplicates. During 2.5 hrs incubation at room temperature on a microplate shaker set to 650 rpm, any c-Kit present is bound to the immobilized capture antibody on the microtiter plate. During washing step (4×300 μL), unbound substances are removed from the plate before 100 μl of an enzyme-linked monoclonal antibody specific for c-Kit is added to the wells. Following 1 h incubation on a shaker and another washing step to remove any unbound detection antibody, 100 μL of substrate solution is added to the plate. Within the next 10 min, the color develops in proportion to the amount of c-Kit bound in the initial step. Color development is stopped by addition of 50 μL stop solution and colour intensity is measured with a plate reader at 450 nm for detection and 570 nm for background subtraction. For generation of calibrator curves, lyophilized, recombinant c-Kit delivered with the kit was reconstituted and diluted in calibrator diluent. The calibration range of the assay is 6.14 pg/mL to 1500 μg/mL. Calibrator 7 (1500 μg/mL) is prepared by 6-fold dilution of stock solution in calibrator diluent and calibrator 6 to calibrator 1 (6.14 pg/mL) are prepared by serial 2.5-fold dilution steps in calibrator diluent. Pure calibrator diluent serves as blank (0 pg/mL). The calibration curves were fitted using a 4-parameter nonlinear regression (Newton/Raphson) with no weighting.

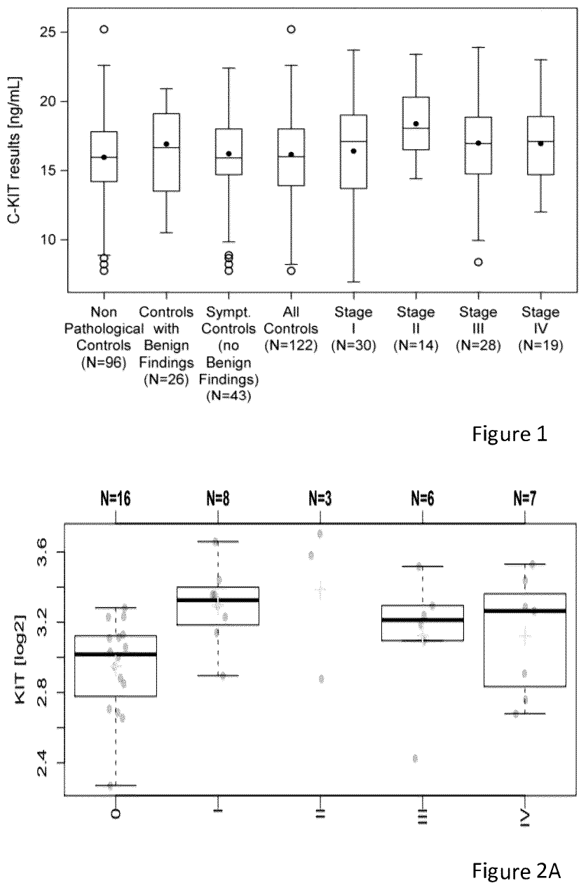

Results are shown in FIG. 1.

Serum c-Kit levels increase gradually in stage I and stage II of endometriosis compared to non-pathological controls and decrease in stage III.

In the following Table 1 the model performance is determined by looking at the area under the curve (AUC). The best possible AUC is 1 while the lowest possible is 0.5. Optimal cut-offs were selected using Youden's index (maximized sum of sensitivity plus specificity-1).

TABLE 1
The diagnostic performance of c-Kit biomarker to discriminate women
with histologically confirmed endometriosis and women without
endometriosis (controls) using receiver operator characteristic
(ROC) analysis is shown describing the area under the curve (AUC)
of the ROC analysis and the associated 95% confidence interval.
N depicts the number of samples tested (cases plus controls, numbers
in each group vary depending on the analyte).

| | AUC (area | 95% CI | | |
|---|---|---|---|---|
| | under the | (confidence | N (sample | type |
| Group comparison | curve) | interval) | size) | Sample |
| symptomatic controls | 0.5616 | 0.4218- | 73 | Serum |
| (w/o benign | | 0.7015 | | |
| pathologies) vs. endo | | | | |
| Stage I | | | | |
| symptomatic controls | 0.7018 | 0.5419- | 57 | Serum |
| (w/o benign | | 0.8607 | | |
| pathologies) vs. endo | | | | |
| Stage II | | | | |
| symptomatic controls | 0.5934 | 0.4551- | 71 | Serum |
| (w/o benign | | 0.7318 | | |
| pathologies) vs. endo | | | | |
| Stage III | | | | |
| symptomatic controls | 0.5906 | 0.4337- | 62 | Serum |
| (w/o benign | | 0.7474 | | |
| pathologies) vs. endo | | | | |
| Stage IV | | | | |
| symptomatic controls | 0.6062 | 0.4851- | 87 | Serum |
| (w/o benign | | 0.7274 | | |
| pathologies) vs. endo | | | | |
| Stage I-II | | | | |
| symptomatic controls | 0.5923 | 0.4735- | 90 | Serum |
| (w/o benign | | 0.7110 | | |
| pathologies) vs. endo | | | | |
| Stage III-IV | | | | |
| Non-pathological | 0.5738 | 0.4480- | 126 | Serum |
| controls vs. endo | | 0.6996 | | |
| Stage I | | | | |
| Non-pathological | 0.7202 | 0.5810- | 110 | Serum |
| controls vs. endo | | 0.8594 | | |
| Stage II | | | | |
| Non-pathological | 0.6062 | 0.4830- | 124 | Serum |
| controls vs. endo | | 0.7294 | | |
| Stage III | | | | |
| Non-pathological | 0.6042 | 0.4616- | 115 | Serum |
| controls vs. endo | | 0.7467 | | |
| Stage IV | | | | |
| Non-pathological | 0.6042 | 0.5165- | 140 | Serum |
| controls vs. endo | | 0.7242 | | |
| Stage I-II | | | | |
| Non-pathological | 0.6054 | 0.5053- | 143 | Serum |
| controls vs. endo | | 0.7055 | | |
| Stage III-IV | | | | |

### Example 2: Diagnostic Performance of Biomarker c-Kit Compared to CA-125 in Women with Endometriosis

In a further experiment the performance of the biomarker c-Kit was compared to the current standard biomarker CA-125.

The concentration of CA-125 was determined by a cobas e 601 analyzer. Detection of CA 125 II with a cobas e 601 analyzer is based on the Elecsys® Electro-Chemiluminescence (ECL) technology. In brief, biotin-labelled and ruthenium-labelled antibodies are combined with the respective amount of undiluted sample and incubated on the analyzer. Subsequently, streptavidin-coated magnetic microparticles are added and incubated on the instrument in order to facilitate binding of the biotin-labelled immunological complexes. After this incubation step the reaction mixture is transferred into the measuring cell where the beads are magnetically captured on the surface of an electrode. ProCell M Buffer containing tripropylamine (TPA) for the subsequent ECL reaction is then introduced into the measuring cell in order to separate bound immunoassay complexes from the free remaining particles. Induction of voltage between the working and the counter electrode then initiates the reaction leading to emission of photons by the ruthenium complexes as well as TPA. The resulting electrochemiluminescent signal is recorded by a photomultiplier and converted into numeric values indicating concentration level of the respective analyte.

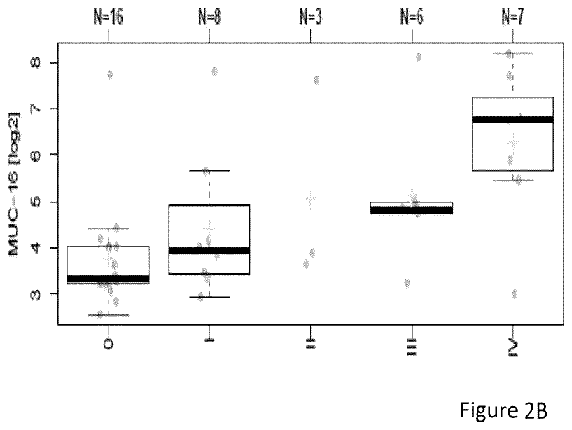

The results are shown in FIGS. 2A and 2B.

Box plots in FIGS. 2A and 2B were generated for controls and for each of the endometriosis Stages (Stage I, Stage II, Stage III, Stage IV) using the data from the high-throughput, multiplex immunoassay-PCR (OLINK proteomics) analysis. The data are presented using box and whisker plots, including the median (middle quartile), the inter-quartile range (which represents the middle 50% of scores for the group), the upper quartile (75% of scores fall below the upper quartile), the lower quartile (25% of scores fall below the lower quartile). The whiskers show the 5th percentile and the 95th percentile, respectively.

Serum c-Kit shows better diagnosis performance for the detection of early stages of endometriosis (Stage I, Stage II) compared to the reference biomarker CA-125.

In the following Table 2 the performance of c-Kit as biomarker compared to CA-125 is determined by looking at the area under the curve (AUC) of both biomarkers for early stage endometriosis (Stage I and II).

TABLE 2
Diagnostic performance of serum c-Kit biomarker and reference
biomarker CA-125 in women with endometriosis (stage I
and stage II) and controls. The measurement of CA-125
was performed by OLINK using the proximity extension assay
(PEA). C-Kit levels were measured using CD117/c-Kit Quantikine
ELISA Kit. C-Kit levels increase gradually in controls
with benign findings, stage I and stage II of endometriosis
and decrease back in stage III and IV.

| | AUC (area | 95% CI | N | |
|---|---|---|---|---|
| Group | under the | (confidence | (sample | Sample |
| comparison | curve) | interval) | size) | type |
| C-Kit controls vs. | 0.5738 | 0.4480- | 126 | Serum |
| Endometriosis | | 0.6996 | | |
| Stage 1 | | | | |
| CA-125 controls vs. | 0.4662 | 0.3499- | 155 | Serum |
| Endometriosis | | 0.5825 | | |
| Stage I | | | | |
| C-Kit controls vs. | 0.7202 | 0.5810- | 110 | Serum |
| Endometriosis | | 0.8594 | | |
| Stage II | | | | |
| CA-125 controls vs. | 0.6002 | 0.4340- | 138 | Serum |
| endometriosis | | 0.7665 | | |
| Stage II | | | | |

The AUC value of the ROC analysis for stage I and stage II endometriosis versus controls was AUC=0.72\AUC=0.57 for serum c-Kit versus AUC=0.46\ AUC=0.6 for serum CA-125.

Serum c-Kit shows better diagnosis performance for the detection of early stages of endometriosis (Stage I, Stage II) compared to the reference biomarker CA-125.

### Clinical Data on Cohort:

1) Endometriosis Cases:
a) Total cohort number: 101 patients
b) Age:

| | No of |
|---|---|
| Age | patients |
| 18-24 | 1 |
| 25-30 | 22 |
| 31-35 | 26 |
| 36-40 | 31 |
| 41-45 | 15 |
| 46-50 | 5 |
| 51-55 | 1 |

c) Hormonal Contraceptives/Devices:

| Hormonal | |
|---|---|
| Contraceptives/ | No of |
| Devices | patients |
| No | 58 |
| Yes | 42 |
| No data | 1 |

No contraceptive hormones/devices: 57.42%
Taking contraceptive hormones/devices: 41.58%
d) Type of Endometriosis and Associated Disorders:

| Endo type/ | No of patients |
|---|---|
| Peritoneal | 81 |
| Endometriosis | |
| Adenomyosis: | 22 |
| Endometrioma: | 6 |
| Deep infiltrating | 5 |
| Endometriosis: | |

e) Endometriosis stage:

| | No of |
|---|---|
| Stage | patients |
| Stage I: | 33 |
| Stage II: | 13 |
| Stage III | 26 |
| Stage VI: | 19 |

f) Menstrual Pain Score:

Menstrual Pain

No data: 38
Mens Pain VAS = 10: 7
Mens Pain VAS = 9: 11
Mens Pain VAS = 8: 11
Mens Pain VAS = 7: 13
Mens Pain VAS = 6: 6
Mens Pain VAS = 5: 3
Mens Pain VAS = 4: 2
Mens Pain VAS = 3: 5
Mens Pain VAS = 2: 3
Mens Pain VAS = 1: 1
Mens Pain VAS = 0: 1

88.11% of patients has (VAS ≥5)

2) No Endometriosis Cases, Severe Pain (“symptomatic controls”):
a) Total cohort number: 65 patients
b) Age:

| | No of |
|---|---|
| Age | patients |
| 18-24 | 13 |
| 25-30 | 14 |
| 31-35 | 10 |
| 36-40 | 15 |
| 41-45 | 8 |
| 46-50 | 5 |
| 51-55 | 0 |

number of patients at Menopausal age (45-50): 6
c) Hormonal Contraceptives/Devices:

| Hormonal | |
|---|---|
| Contraceptives/ | No of |
| Devices | patients |
| No | 42 |
| Yes | 21 |
| No data | 2 |

No contraceptive hormones/devices: 64.61%
Taking contraceptive hormones/devices: 32.30%
d) Menstrual Pain Score:

Menstrual Pain Score

No data: 0
Mens Pain VAS = 10: 9
Mens Pain VAS = 9: 17
Mens Pain VAS = 8: 15
Mens Pain VAS = 7: 24
Mens Pain VAS = 6: 0
Mens Pain VAS = 5: 0
Mens Pain VAS = 4: 0
Mens Pain VAS = 3: 0
Mens Pain VAS = 2: 0
Mens Pain VAS = 1: 0
Mens Pain VAS = 0: 0

100% of patients has (VAS ≥7)

3) No Endometriosis, No Pain (“non-pathologic controls”):
a) Total cohort number: 84 patients
b) Age:

| | No of |
|---|---|
| Age | patients |
| 18-24 | 0 |
| 25-30 | 14 |
| 31-35 | 24 |
| 36-40 | 15 |
| 41-45 | 16 |
| 46-50 | 11 |
| 51-55 | 4 |

c) Hormonal Contraceptives/Devices:

| Hormonal | |
|---|---|
| Contraceptives/ | No of |
| Devices | patients |
| No | 62 |
| Yes | 20 |
| No data | 2 |

No contraceptive hormones/devices: 73.8%
Taking contraceptive hormones/devices: 23.8%
d) Menstrual Pain Score:

Menstrual Pain Score

No data: 0
Mens Pain VAS = 10: 0
Mens Pain VAS = 9: 0
Mens Pain VAS = 8: 0
Mens Pain VAS = 7: 0
Mens Pain VAS = 6: 0
Mens Pain VAS = 5: 0
Mens Pain VAS = 4: 0
Mens Pain VAS = 3: 0
Mens Pain VAS = 2: 23
Mens Pain VAS = 1: 16
Mens Pain VAS = 0: 45

100% of patients has (VAS≤2)

1. An in vitro method for diagnosing endometriosis in a subject, the method comprising the steps of:
a) determining the level of c-Kit in a biological fluid sample from the subject;
b) comparing the level of c-Kit to at least one appropriate reference value of a c-Kit level; and
c) identifying a subject as having endometriosis if the comparison in step b) indicates that the subject has an increased level of c-Kit compared to the appropriate reference value.

2. An in vitro method for classifying the stage of endometriosis in a subject, the method comprising the steps of:
a) determining the level of c-Kit in a biological fluid sample from the subject;
b) comparing the level of c-Kit to at least one appropriate reference value of a c-Kit level; and
c) classifying the stage of endometriosis in the subject if the comparison in step b) indicates that the subject has an increased or decreased level of c-Kit compared to the at least one appropriate reference value of a c-Kit level.

3. The method according to claim 2, wherein the biological fluid sample is blood, serum, plasma, capillary blood, interstitial fluid, peritoneal fluid, or menstrual fluid.

4. The method according to claim 1, wherein the diagnosis or classification is performed independent of the scoring system of the American Society for Reproductive Medicine

5. The method according to claim 2, wherein the subject's stage of endometriosis is classified as stage I, stage II, stage III or stage IV endometriosis according to the revised scoring system of the American Society for Reproductive Medicine (r-ASRM).

6. The method according to claim 1, wherein the subject's stage of endometriosis is classified as stage I or stage II endometriosis according to the revised scoring system of the American Society for Reproductive Medicine (r-ASRM).

7. The method according to claim 1, wherein endometriosis is selected from the group consisting of peritoneal endometriosis, endometrioma, deep infiltrating endometriosis, and adenomyosis.

8. The method according to claim 1, further comprising selecting a treatment regimen for the subject based on the comparison of the level of c-Kit with the control sample or with the pre-determined reference level.

9. The method according to claim 8, further comprising administering the selected treatment regimen to the subject, optionally wherein the selected treatment regimen comprises drug-based therapy and/or surgical treatment (laparoscopy).

10. An in vitro method for monitoring endometriosis progression in a subject, the method comprising the steps of:
i. determining the level of c-Kit in a biological fluid sample from the subject in accordance with method steps a) to b) of claim 1;
ii. repeating step i. using a biological fluid sample obtained from the subject during or after treatment for a time interval; and
iii. comparing the levels of c-Kit identified in i. with the c-Kit levels identified in ii., wherein a change in the c-Kit levels from i. to ii. is indicative of a change in endometriosis progression in the subject.

11. An in vitro method for determining the therapeutic effect of a treatment regimen for endometriosis in a subject, the method comprising the steps of:
i. determining the level of c-Kit in a biological fluid sample from the subject in accordance with method steps a) to b) of claim 1;
ii. repeating step a) using a biological fluid sample obtained from the subject during or after treatment for a time interval; and
iii. comparing the level of c-Kit determined in step a) to that determined in step b), and identifying that the treatment regimen has a therapeutic effect if the level of c-Kit decreased after treatment.

12. The method according to claim 1, further comprising the assessment of dysmenorrhea according to the Visual Analog Scale (VAS) and/or lower abdominal pain according to VAS.

13. The method according to claim 1, further comprising determining the level of CA-125 in the biological fluid sample from the subject.

14. The method according to claim 13, comprising calculating
a ratio of the amount or concentration of c-Kit and the amount or concentration of CA-125, or
a ratio of the amount or concentration of c-Kit and dysmenorrhea, or
a ratio of the amount or concentration of c-Kit and the amount or concentration of CA-125 and dysmenorrhea, or
a ratio of the amount or concentration of c-Kit and lower abdominal pain according to the VAS scale.

15. A computer-implemented method for assessing a patient with suspected endometriosis comprising the steps of:
(a) receiving a value for the level of a first biomarker in a biological fluid sample of the subject, said first biomarker being c-Kit;
(b) receiving a value for the level of a second biomarker in a sample of the subject, wherein said second biomarker is CA125;
(c) receiving a value for the level of dysmenorrhea according to the VAS and/or lower abdominal pain according to the VAS;
(d) comparing the values for the levels of steps (a)-(c) to references for said biomarkers and the amount of dysmenorrhea and/or calculating a score for assessing the subject with suspected endometriosis based on the levels of the biomarkers and the amount of dysmenorrhea; and
(e) assessing said subject based on the comparison and/or the calculation made in step (d).

16. The method according to claim 3, wherein the biological fluid sample is serum.
